(19) United States
(12) Patent Application Publication
(10) Pub. No.: US 2026/0160724 A1
(43) Pub. Date: Jun. 11, 2026

(54) GAS SENSOR

(71) Applicant: TDK Corporation, Tokyo (JP)

(72) Inventor: Yoshio KAITA, Tokyo (JP)

(21) Appl. No.: 19/408,746

(22) Filed: Dec. 4, 2025

(30) Foreign Application Priority Data
Dec. 6, 2024 (JP) 2024-213243

(51) Int. Cl. G01N 27/18; G01N 33/00

(52) U.S. Cl. CPC G01N 27/18; G01N 33/004

(57) ABSTRACT

Disclosed herein is a gas sensor that includes: a substrate having a first cavity; a first heater supported on the substrate so as to overlap the first cavity; a first temperature-sensitive element supported on the substrate so as not to overlap the first heater; and a signal processing circuit configured to heat the first heater during gas concentration measurement. A temperature of the first temperature-sensitive element during the gas concentration measurement changes due to heat conducted from the first heater, mainly through the substrate, and in accordance with a change in a temperature of the first heater.

### CROSS-REFERENCE TO RELATED APPLICATION

This application claims the benefit of Japanese Patent Application No. 2024-213243, filed on December 6, 2024, the entire disclosure of which is incorporated by reference herein.

### TITLE OF THE INVENTION

### BACKGROUND OF THE ART

### Field of the Art

The present disclosure relates to a gas sensor and, more particularly, to a gas sensor using a heater.

### Description of Related Art

International Publication WO 2020/031517 discloses a gas sensor that uses a heater to heat a detection thermistor to 100 to 200°C and another heater to heat a reference thermistor to 250 to 350°C, and uses an output voltage appearing at the node between the detection thermistor and the reference thermistor in this state, thereby enabling measurement of the concentration of a gas to be measured in a measurement atmosphere.

However, in the gas sensor disclosed in International Publication WO 2020/031517, the detection thermistor and the reference thermistor are disposed immediately above their respective heaters, and are therefore heated to high temperatures themselves. This may accelerate aging of both the thermistors.

### SUMMARY

A gas sensor according to an aspect of the present disclosure includes: a substrate having a first cavity; a first heater supported on the substrate so as to overlap the first cavity; a first temperature-sensitive element supported on the substrate so as not to overlap the first heater; and a signal processing circuit configured to heat the first heater during gas concentration measurement, wherein a temperature of the first temperature-sensitive element during the gas concentration measurement changes due to heat conducted from the first heater, mainly through the substrate, and in accordance with a change in a temperature of the first heater.

### BRIEF DESCRIPTION OF THE DRAWINGS

The above features and advantages of the present disclosure will be more apparent from the following description of some embodiments taken in conjunction with the accompanying drawings, in which:

FIG. 1 is a circuit diagram illustrating the configuration of a gas sensor 100 according to a first embodiment of the technology described herein;

FIG. 2 is a schematic plan view illustrating the configuration of the sensor part 10;

FIG. 3A is a schematic cross-sectional view taken along the line A-A’ in FIG. 2;

FIG. 3B is a schematic cross-sectional view taken along the line B-B’ in FIG. 2;

FIG. 4 is a flowchart for explaining the operation of the gas sensor 100 during the gas concentration measurement;

FIG. 5 is a timing chart for explaining the operation of the gas sensor 100. The gas concentration measurement is executed in the period T illustrated in FIG. 5;

FIG. 6 is a schematic plan view illustrating the configuration of a sensor part 10A according to a first modification of the first embodiment;

FIG. 7A is a schematic cross-sectional view taken along the line A-A’ in FIG. 6;

FIG. 7B is a schematic cross-sectional view taken along the line B-B’ in FIG. 6;

FIG. 8 is a schematic plan view illustrating the configuration of a sensor part 10B according to a second modification of the first embodiment;

FIG. 9 is a schematic cross-sectional view taken along the line C-C’ in FIG. 8;

FIG. 10 is a circuit diagram illustrating the configuration of a gas sensor 200 according to a second embodiment of the technology described herein;

FIG. 11 is a table for explaining the function of the multiplexer 27;

FIG. 12 is a flowchart for explaining the operation of the gas sensor 200 during the gas concentration measurement;

FIG. 13 is a timing chart for explaining the operation of the gas sensor 200;

FIG. 14 is a schematic plan view illustrating the configuration of the sensor part 30;

FIG. 15 is a schematic plan view illustrating the configuration of a sensor part 30A according to a first modification of the second embodiment;

FIG. 16 is a schematic plan view illustrating the configuration of a sensor part 30B according to a second modification of the second embodiment;

FIG. 17 is a schematic cross-sectional view taken along the line A-A’ in FIG. 16;

FIG. 18 is a schematic plan view illustrating the configuration of a sensor part 30C according to a third modification of the second embodiment;

FIG. 19 is a circuit diagram illustrating the configuration of a gas sensor 300 according to a third embodiment of the technology described herein;

FIG. 20 is a table for explaining the function of the multiplexer 28;

FIG. 21 is a circuit diagram illustrating the configuration of a gas sensor 400 according to a fourth embodiment of the technology described herein;

FIG. 22 is a schematic plan view illustrating the configuration of the sensor part 50;

FIG. 23 is a schematic cross-sectional view taken along the line D-D’ in FIG. 22;

FIG. 24 is a circuit diagram illustrating the configuration of a gas sensor 500 according to a fifth embodiment of the technology described herein; and

FIG. 25 is a schematic plan view illustrating the configurations of the sensor part 70 and the temperature sensor 80.

### DETAILED DESCRIPTION OF THE EMBODIMENTS

The present disclosure describes a technology for suppressing aging of a temperature-sensitive element, such as a thermistor, used in a gas sensor with a heater.

Some embodiments of the present disclosure will be explained below in detail with reference to the accompanying drawings.

### <First Embodiment>

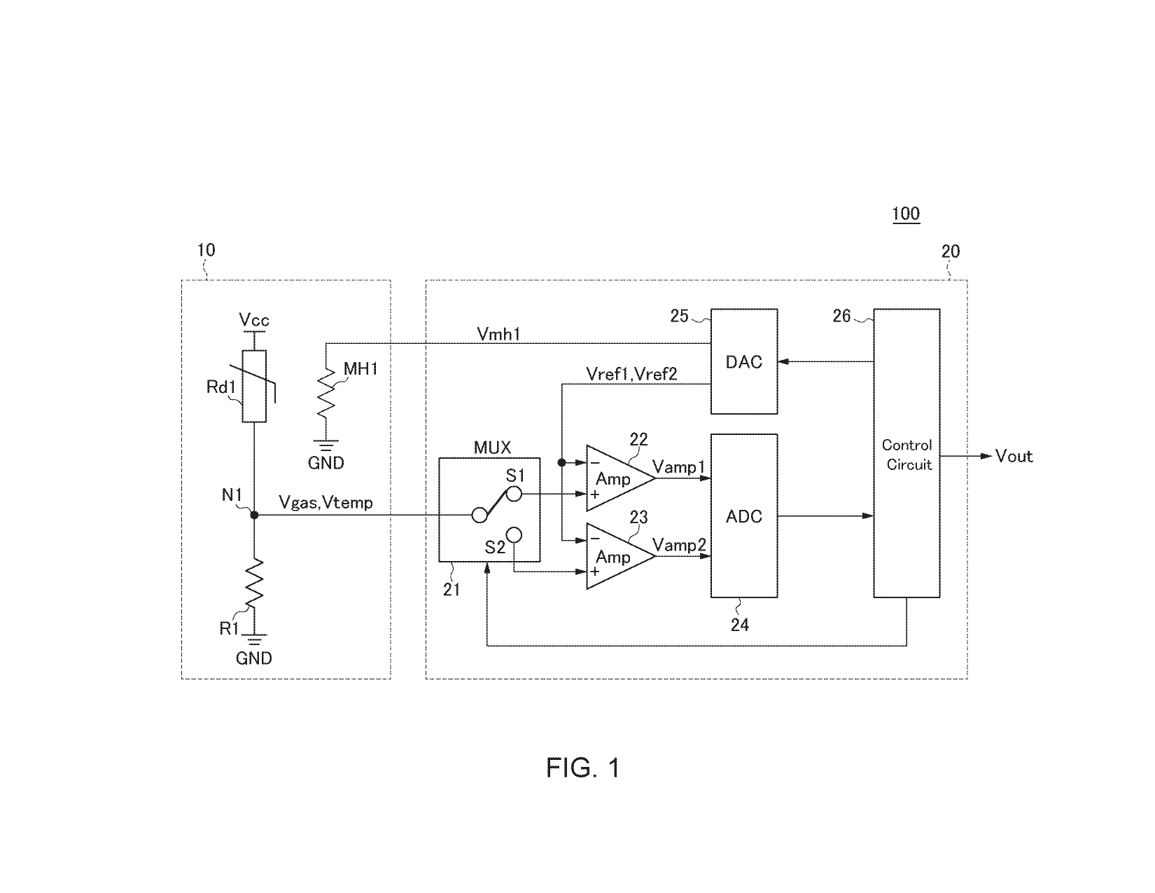

FIG. 1 is a circuit diagram illustrating the configuration of a gas sensor 100 according to a first embodiment of the technology described herein.

As illustrated in FIG. 1, the gas sensor 100 according to the first embodiment includes a sensor part 10 and a signal processing circuit 20. Although not particularly limited, the gas sensor 100 according to the first embodiment is a heat-conduction type gas sensor for detecting the concentration of CO₂ gas in a measurement atmosphere.

The sensor part 10 includes a thermistor Rd1 and a fixed resistor R1, which are connected in series in this order between a power supply Vcc and a ground GND, and further includes a heater MH1. The thermistor Rd1 changes its temperature in response to a change in the temperature of the heater MH1. The thermistor Rd1 is a temperature-sensitive element whose resistance value varies with temperature. As described later, at a node N1 between the thermistor Rd1 and the fixed resistor R1, a temperature detection signal Vtemp appears during a temperature detection period, while a gas detection signal Vgas appears during a gas detection period.

During the gas detection period, the heater MH1 is heated to a first temperature range. The first temperature range refers to a predetermined range, for example, from 100°C to 230°C, or around 170°C. The term “temperature range” in the present disclosure refers to a range having, for example, a width of 1°C or less. Thus, the temperature range around 170°C may be from 169.5°C to 170.5°C, for example.

In the first temperature range, the thermal conductivity of CO₂ gas is lower than that of air. Therefore, when CO₂ gas is present in the measurement atmosphere in a state in which a fixed power is applied to the heater MH1 to heat it to the first temperature range, the temperature of the heater MH1 rises as the concentration of CO₂ gas increases. As a result, the temperature of the thermistor Rd1 also rises. Assume that the heater MH1 is heated to 170°C under the condition that the CO₂ gas concentration in the measurement atmosphere is at the normal atmospheric level (e.g., 400 ppm). In this case, when the CO₂ gas concentration in the measurement atmosphere exceeds the normal level, the temperature of the heater MH1 rises above 170°C depending on the CO₂ gas concentration, and the temperature of the thermistor Rd1 also becomes higher than that in the case of normal atmospheric CO₂ gas concentration. As a result, when the thermistor Rd1 has a negative temperature coefficient of resistance (i.e., when it is an NTC thermistor), the resistance value of the thermistor Rd1 decreases as the CO₂ gas concentration in the measurement atmosphere increases. Thus, when CO₂ gas is present in the measurement atmosphere in a state in which the heater MH1 is heated to the first temperature range, the heat dissipation characteristics of the heater MH1 change in accordance with the concentration of the CO₂ gas. This change appears as a change in the temperature of the thermistor Rd1, i.e., a change in the resistance value thereof.

The thermistor Rd1 and the fixed resistor R1 are connected in series between the power supply Vcc and the ground GND, so that when the thermistor Rd1 has a negative temperature coefficient of resistance, the level of the gas detection signal Vgas appearing at the node N1 increases with the CO₂ gas concentration in the measurement atmosphere.

The signal processing circuit 20 includes a multiplexer (MUX) 21, differential amplifiers 22 and 23, an AD converter (ADC) 24, a DA converter (DAC) 25, and a control circuit 26.

The multiplexer 21 is a circuit configured to connect the node N1 included in the sensor part 10 to one of the selection nodes S1 and S2, and the selection operation thereof is controlled by the control circuit 26. The differential amplifier 22 is configured to compare the gas detection signal Vgas appearing at the selection node S1 with a reference potential Vref1 to generate an amplification signal Vamp1 corresponding to the amplified level difference (= Vgas – Vref1) between the gas detection signal Vgas and the reference potential Vref1. The differential amplifier 23 is configured to compare the temperature detection signal Vtemp appearing at the selection node S2 with a reference potential Vref2 to generate an amplification signal Vamp2 corresponding to the amplified level difference (= Vtemp – Vref2) between the temperature detection signal Vtemp and the reference potential Vref2. The amplification signals Vamp1 and Vamp2 are input to the AD converter 24. The AD converter 24 AD-converts the amplification signals Vamp1 and Vamp2 into corresponding digital values and supplies them to the control circuit 26.

The control circuit 26 calculates the concentration of CO₂ gas, which is a gas to be measured, based on the AD-converted amplification signal Vamp1 and generates an output signal Vout indicating the CO₂ gas concentration. The output signal Vout is output outside the gas sensor 100. The control circuit 26 may calculate the CO₂ gas concentration using a calculation formula set therein. Further, the control circuit 26 supplies digital values of various control parameters to the DA converter 25. The DA converter 25 DA-converts the digital values of the various control parameters to generate a heater voltage Vmh1 and the reference potentials Vref1 and Vref2. The heater voltage Vmh1 is applied to the heater MH1 for heating. The reference potentials Vref1 and Vref2 are supplied to the differential amplifiers 22 and 23, respectively. The reference potentials Vref1 and Vref2 may have the same level.

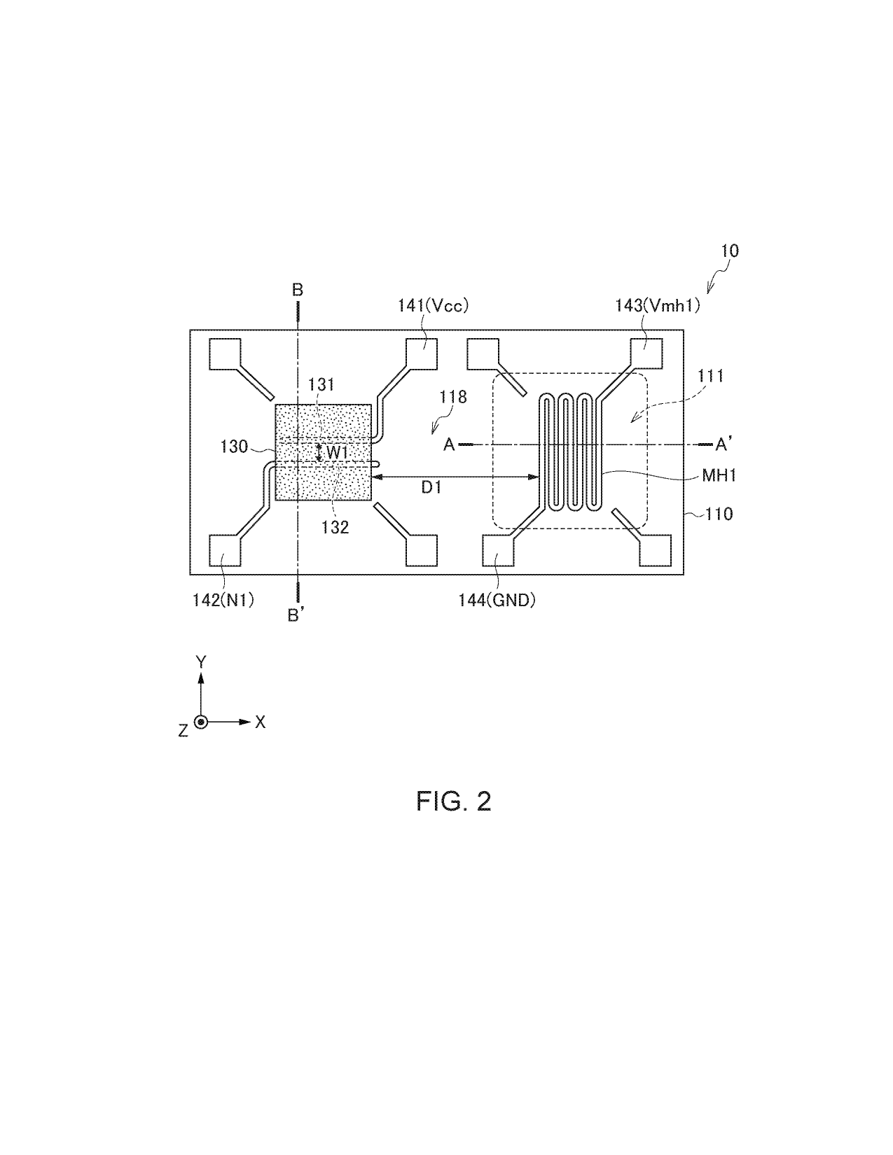

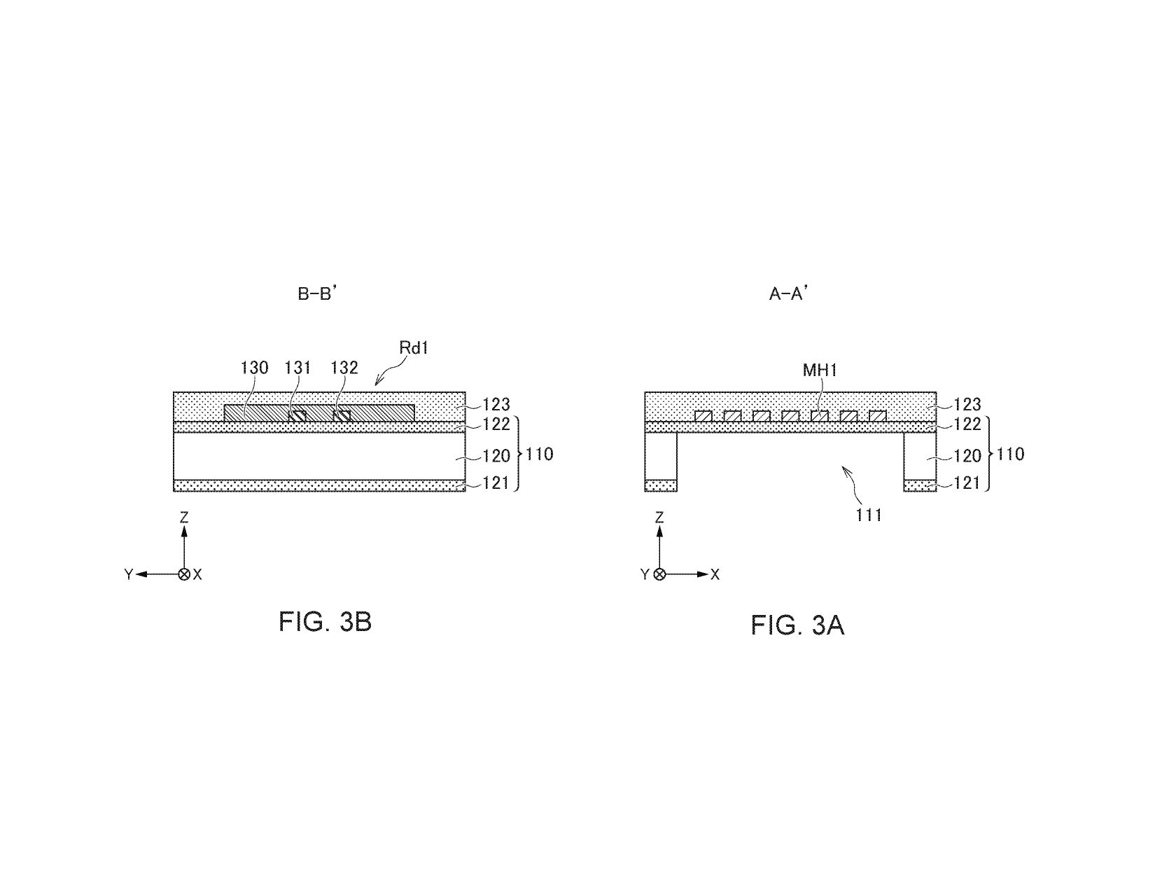

FIG. 2 is a schematic plan view illustrating the configuration of the sensor part 10. FIG. 3A is a schematic cross-sectional view taken along the line A-A’ in FIG. 2, and FIG. 3B is a schematic cross-sectional view taken along the line B-B’ in FIG. 2.

As illustrated in FIGS. 2, 3A, and FIG. 3B, the sensor part 10 includes a substrate 110 composed of a main body 120 and insulating films 121 and 122 formed on the lower and upper surfaces of the main body 120, respectively, the heater MH1 and thermistor electrodes 131 and 132 which are provided on the insulating film 122, a thermistor resistor 130 that covers the pair of thermistor electrodes 131 and 132, and an insulating film 123 that covers the heater MH1 and the thermistor resistor 130. The thermistor resistor 130 and the pair of thermistor electrodes 131 and 132 constitute the thermistor Rd1 illustrated in FIG. 1.

The substrate 110 serves as a support for supporting the heater MH1 and thermistor Rd1. The main body 120 of the substrate 110 is not particularly limited in material as long as it has adequate mechanical strength and is suitable for fine processing such as etching. Examples of the material of the substrate 110 include a silicon substrate, a sapphire substrate, a ceramic substrate, a quartz substrate, and a glass substrate. The substrate 110 has a cavity 111 at a position overlapping the heater MH1 in a plan view as seen from the Z-direction so as to enhance the heat efficiency of the heater MH1. In the area where the cavity 111 is formed, the insulating film 121 of the substrate 110 is removed, and the thickness of the main body 120 of the substrate 110 in the Z-direction is locally reduced, or both the main body 120 of the substrate 110 and insulating film 121 are removed. In the example illustrated in FIG. 3A, both the main body 120 of the substrate 110 and insulating film 121 are removed in the area where the cavity 111 is formed, whereby the heater MH1 is supported on the substrate 110 through the insulating films 122 and 123.

The insulating films 121 to 123 may be made of an inorganic insulating material, such as silicon oxide or silicon nitride. The heater MH1 has a meandered wire structure formed of a metal material having a relatively high melting point, such as molybdenum (Mo), platinum (Pt), gold (Au), tungsten (W), tantalum (Ta), palladium (Pd), iridium (Ir), or an alloy thereof. One end of the heater MH1 is connected to a terminal electrode 143 that receives the heater voltage Vmh1, and the other end is connected to a terminal electrode 144 that receives the ground potential GND.

The thermistor resistor 130 is made of a material whose resistance value varies with temperature, such as vanadium oxide, amorphous silicon, polycrystalline silicon, an oxide with a spinel crystal structure containing manganese, titanium oxide, or yttrium-barium-copper oxide. The pair of thermistor electrodes 131 and 132 are in contact with the thermistor resistor 130. Thus, the resistance value between the thermistor electrodes 131 and 132 is defined by the resistance value of the thermistor resistor 130 located between the electrodes. The distance W1 between the thermistor electrodes 131 and 132 is, for example, 5 to 8 µm, and may be selected according to the target resistance value of the thermistor Rd1. The thermistor electrode 131 is connected to a terminal electrode 141 that receives the power supply potential Vcc, and the thermistor electrode 132 is connected to a terminal electrode 142 that constitutes the node N1.

As illustrated in FIG. 2, in the present embodiment, the thermistor Rd1 is disposed so as not to overlap the heater MH1. That is, in the present embodiment, the heater MH1 and the thermistor Rd1 are located at different positions in the plane. In the example illustrated in FIG. 2, their positions differ in the X-direction. Thus, the thermistor Rd1 is not heated directly by the heater MH1, but is mainly heated by thermal conduction through the substrate 110. In the present embodiment, the heater MH1 and the thermistor Rd1 are spaced apart from each other with a portion (first portion 118) of the substrate 110 interposed therebetween. The thickness of the first portion 118 of the substrate 110 in the Z-direction is larger than that of a portion of the substrate 110 that overlaps the cavity 111 in the Z-direction. Further, in the present embodiment, the substrate 110 has no cavity at the position overlapping the thermistor Rd1.

With such a configuration, in the case where the heater MH1 is heated to 170°C under the condition that the CO₂ gas concentration in the measurement atmosphere is at the normal atmospheric level (e.g., 400 ppm), when the CO₂ gas concentration in the measurement atmosphere exceeds the normal level, the temperature of the heater MH1 rises above 170°C depending on the CO₂ gas concentration. Heat from the heater MH1 is conducted to the thermistor Rd1 mainly through the substrate 110. Accordingly, the temperature of the thermistor Rd1 also becomes higher than that in the case of normal atmospheric CO₂ gas concentration (e.g., 400 ppm). As a result, when the thermistor Rd1 has a negative temperature coefficient of resistance (i.e., when it is an NTC thermistor), the resistance value of the thermistor Rd1 decreases as the CO₂ gas concentration in the measurement atmosphere increases.

As described above, in the present embodiment, the temperature of the thermistor Rd1 changes due to heat conducted from the heater MH1, mainly through the substrate 110, and in accordance with a change in the temperature of the heater MH1, so that the temperature rise of the thermistor Rd1 is significantly suppressed compared with the case where the thermistor Rd1 is disposed immediately above or below the heater MH1. For example, when the heater MH1 is heated from room temperature to 170°C, the temperature rise of the thermistor Rd1 is limited to only a few degrees above room temperature. The temperature of the thermistor Rd1 during heating of the heater MH1 can be controlled by adjusting the distance D1 between the thermistor resistor 130 and the heater MH1.

The following describes the operation of the gas sensor 100 according to the first embodiment during gas concentration measurement.

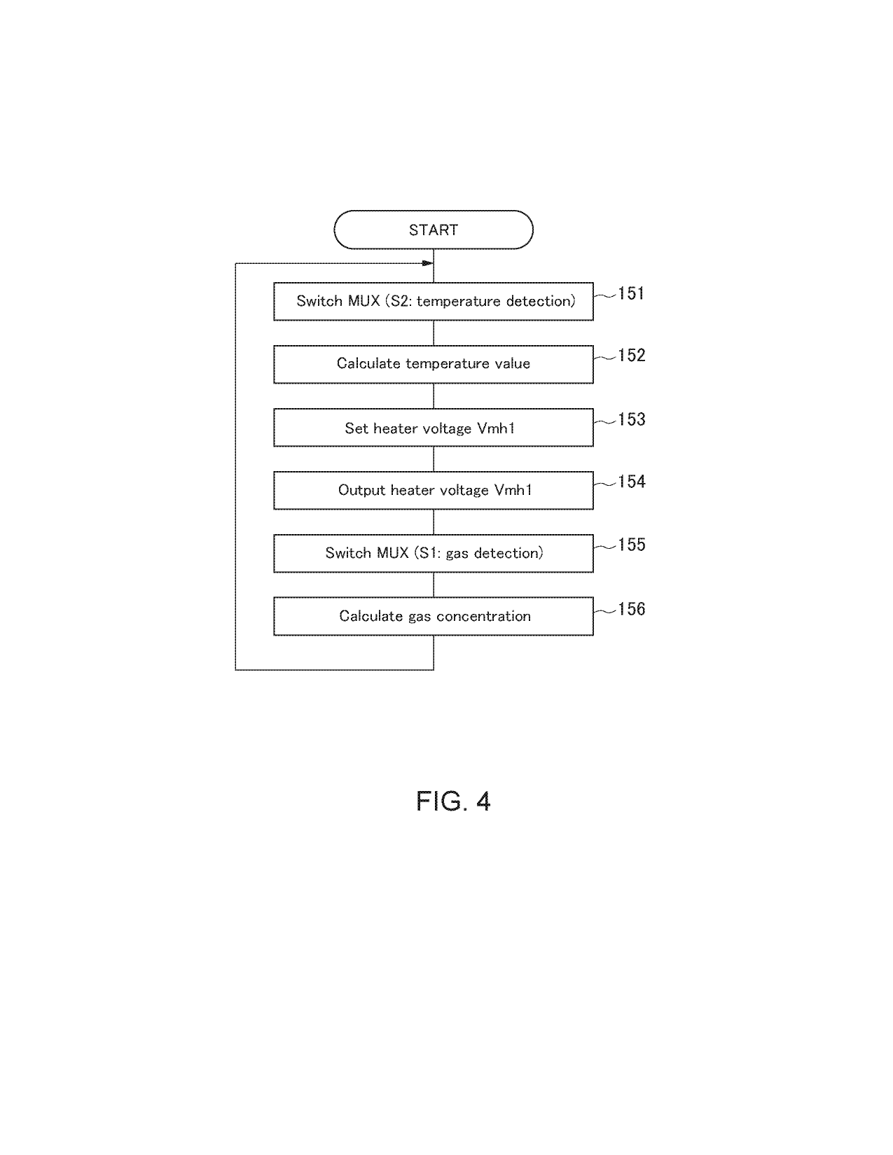

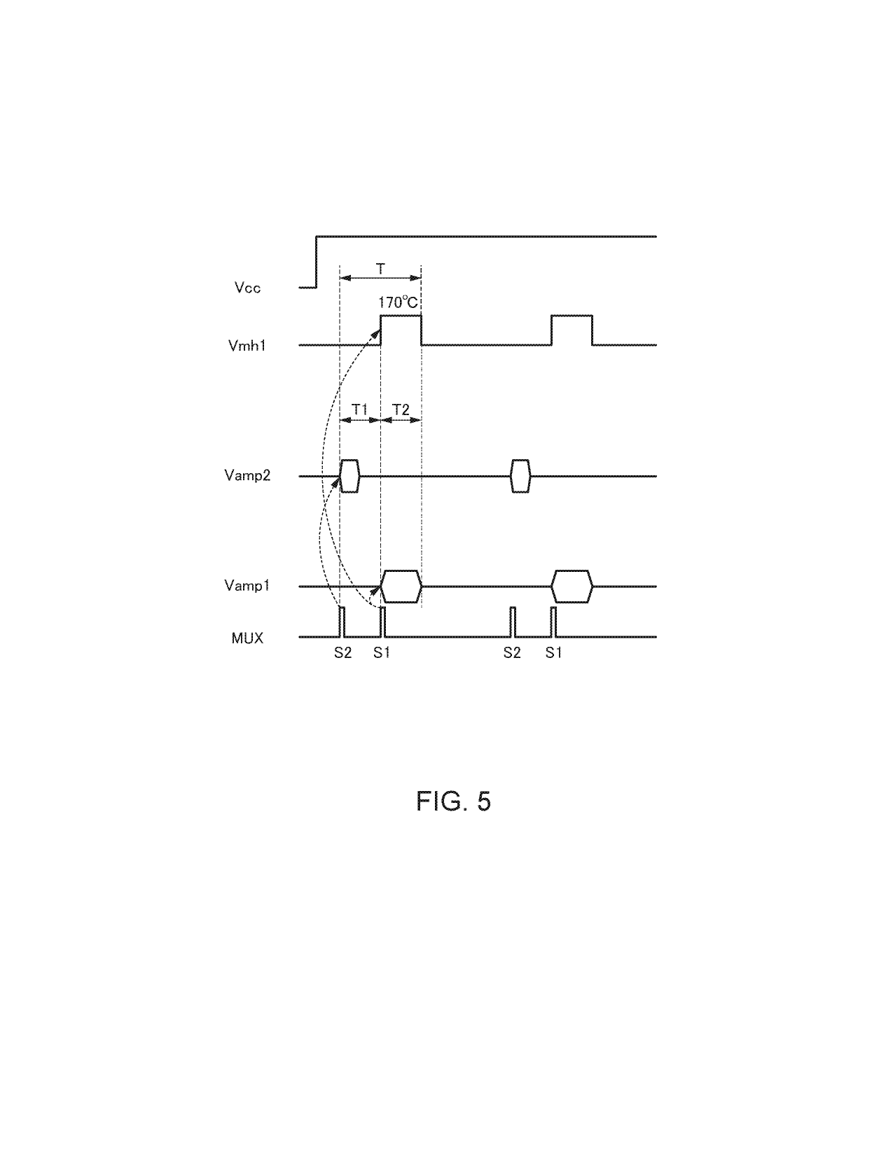

FIG. 4 is a flowchart for explaining the operation of the gas sensor 100 during the gas concentration measurement. FIG. 5 is a timing chart for explaining the operation of the gas sensor 100. The gas concentration measurement is executed in the period T illustrated in FIG. 5.

The control circuit 26 included in the signal processing circuit 20 controls the multiplexer 21 to select the selection node S2 and supplies the reference potential Vref2 to the differential amplifier 23 through the DA converter 25 (step 151). As a result, the temperature detection signal Vtemp indicating the current temperature of the thermistor Rd1 is generated. At this time, the heater voltage Vmh1 is not supplied to the heater MH1, so that the temperatures of the thermistor Rd1 and heater MH1 are maintained at the ambient temperature, allowing the temperature detection signal Vtemp to be regarded as a signal indicating the ambient temperature. The ambient temperature refers to the temperature of the measurement environment. Therefore, in this state, the temperature detection signal Vtemp appearing at the node N1 reflects the current ambient temperature. The temperature detection signal Vtemp is converted into the amplification signal Vamp2 by the differential amplifier 23 and thereafter converted into a corresponding digital value by the AD converter 24. Subsequently, the control circuit 26 calculates the current ambient temperature based on the AD-converted amplification signal Vamp2 (step 152). These operations are executed in the temperature detection period T1 illustrated in FIG. 5. The temperature detection period T1 corresponds to the first half of the gas concentration measurement period T.

Then, the control circuit 26 calculates the heater voltage Vmh1 based on the ambient temperature that has been calculated based on the amplification signal Vamp2 (step 153). For example, when the CO₂ gas concentration in the measurement atmosphere is at the normal atmospheric level (e.g., 400 ppm) irrespective of the current ambient temperature, the level of the heater voltage Vmh1 is set so as to heat the heater MH1 to 170°C. Step 152 may be omitted, and in step 153, the heater voltage Vmh1 may be calculated directly based on the amplification signal Vamp2. The heater voltage Vmh1 thus calculated is supplied to the heater MH1 (step 154).

In this state, the control circuit 26 controls the multiplexer 21 to select the selection node S1 and supplies the reference potential Vref1 to the differential amplifier 22 through the DA converter 25 (step 155). At the node N1, the gas detection signal Vgas having a level obtained by dividing the power supply VCC with the resistance value of the thermistor Rd1 and that of the fixed resistor R1 appears. The resistance value of the thermistor Rd1 is influenced both by heat from the heater MH1 conducted through the substrate 110 and by the ambient temperature. Thus, the control circuit 26 adjusts the reference potential Vref1 based on the ambient temperature (indicated by the amplification signal Vamp2), in order to prevent the amplification signal Vamp1 from being affected by the ambient temperature. The calculation of the reference potential Vref1 based on the ambient temperature can be performed by the following method, for example. First, with CO₂ gas concentration maintained at the normal atmospheric level (e.g., 400 ppm), the gas detection signal Vgas is measured while varying the ambient temperature. The heater voltage Vmh1 is set to a level that heats the heater MH1 to, for example, 170°C, irrespective of the ambient temperature. As a result, the relationship between the ambient temperature (amplification signal Vamp2) and the gas detection signal Vgas in the state in which CO₂ gas concentration is maintained at the normal atmospheric level (e.g., 400 ppm) is identified. Then, the level of the gas detection signal Vgas corresponding to each ambient temperature (amplification signal Vamp2) is determined as the level of the reference potential Vref1 at that ambient temperature (amplification signal Vamp2). The relationship between the ambient temperature (amplified signal Vamp2) and the reference potential Vref1 may be stored in the control circuit 26 in the form of an approximate expression.

The gas detection signal Vgas is converted into the amplification signal Vamp1 by the differential amplifier 22. The control circuit 26 calculates CO₂ gas concentration based on the AD-converted amplification signal Vamp1 and outputs the output signal Vout indicating the CO₂ gas concentration (step 156). The operations from steps 153 to 156 are executed in the gas detection period T2 illustrated in FIG. 5. The gas detection period T2 corresponds to the latter half of the gas concentration measurement period T.

Such periodic gas concentration measurement makes it possible to periodically detect a change in the concentration of CO₂ gas in the measurement atmosphere.

As described above, in the gas sensor 100 according to the present embodiment, the thermistor Rd1 and the heater MH1 are arranged at different locations on the substrate 110 so as not to overlap each other. As a result, the temperature of the thermistor Rd1 during the gas concentration measurement is sufficiently lower than the heating temperature of the heater MH1, making it possible to suppress aging of the thermistor Rd1 caused by exposure to high temperatures.

In addition, In the present embodiment, the temperature rise of the thermistor Rd1 caused by heating of the heater MH1 is significantly suppressed. As a result, the temperature difference of the thermistor Rd1 between the temperature detection period T1 and the gas detection period T2 is small. This allows the thermistor Rd1 to be used for both gas concentration detection and temperature detection, thereby eliminating the need for an additional thermistor for temperature detection, as is required in a typical gas sensor.

Further, in the present embodiment, since no cavity is formed in the substrate 110 immediately below the thermistor Rd1, the heat capacity in the vicinity of the thermistor Rd1 is large. Accordingly, by setting a relatively long heating time for the heater MH1, the temperature of the thermistor Rd1 can be stabilized, making it possible to measure CO₂ gas concentration more accurately.

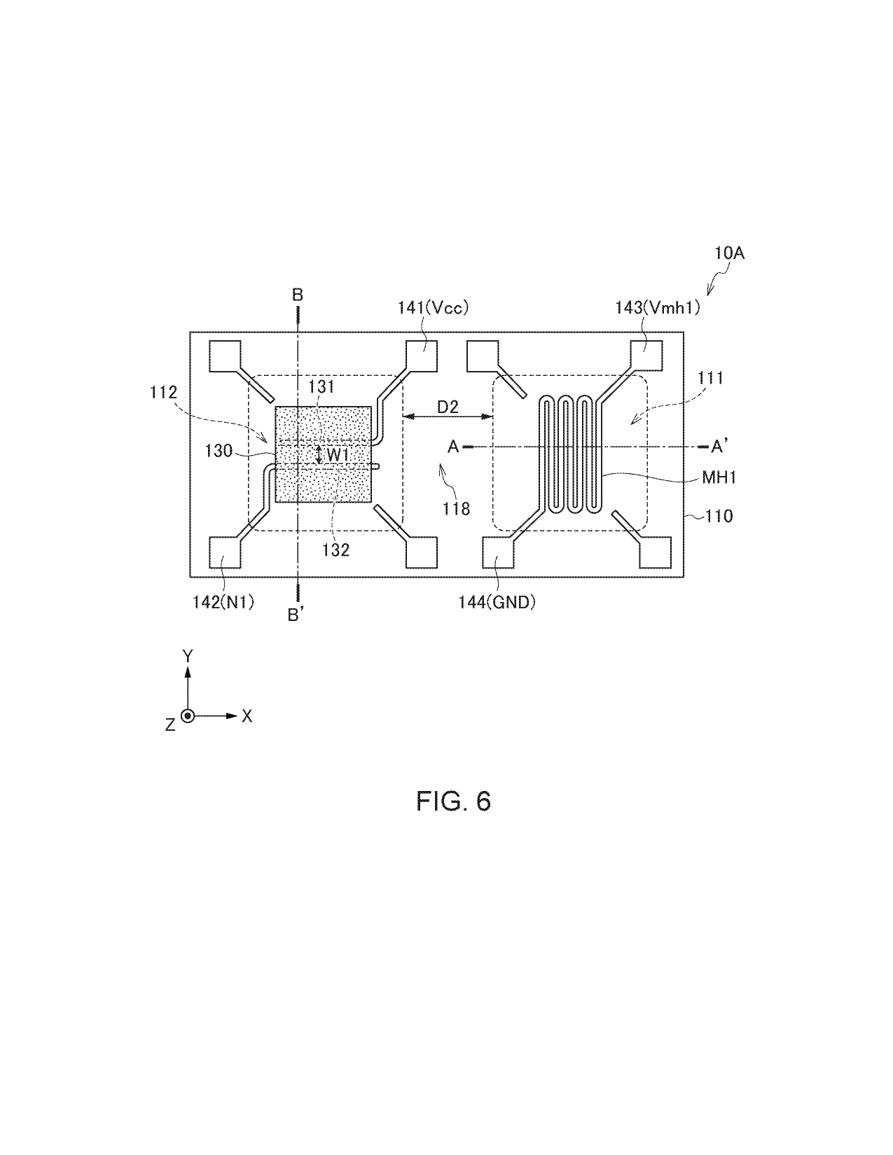

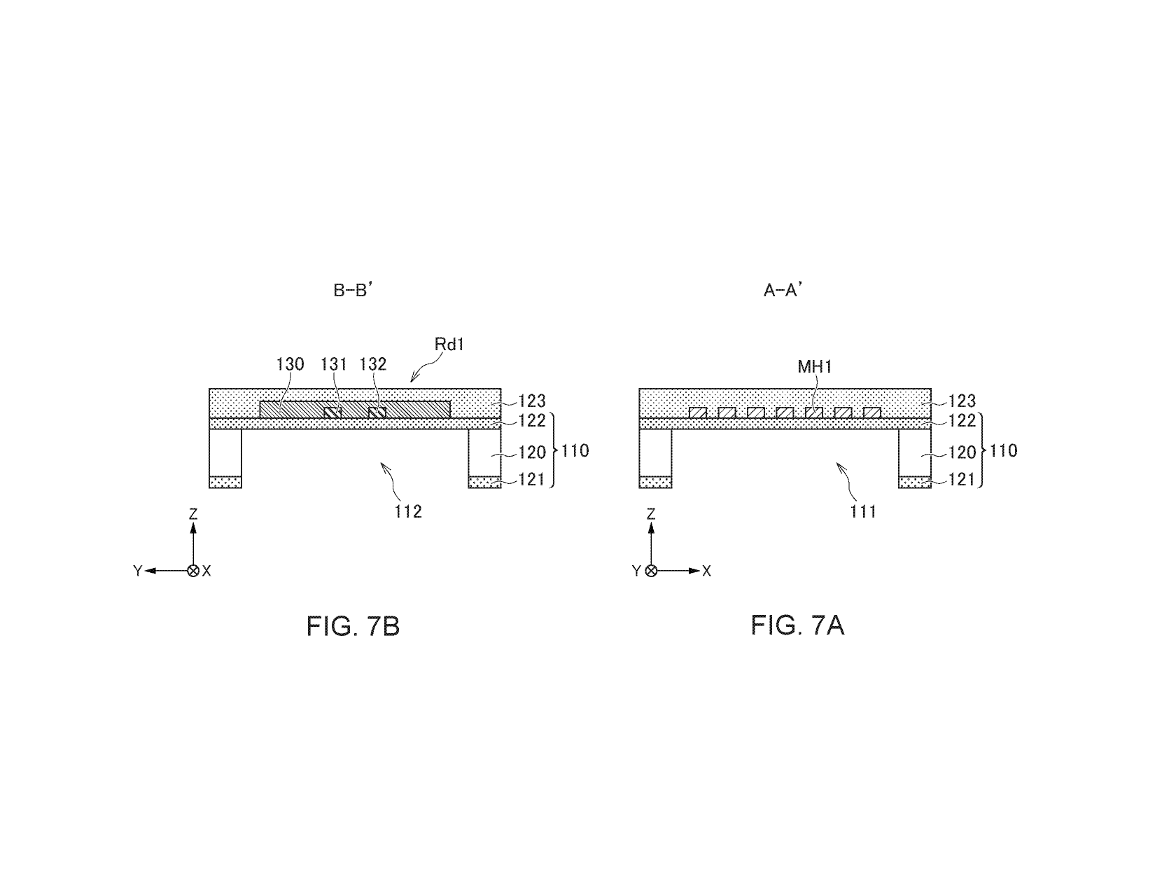

FIG. 6 is a schematic plan view illustrating the configuration of a sensor part 10A according to a first modification of the first embodiment. FIG. 7A is a schematic cross-sectional view taken along the line A-A’ in FIG. 6, and FIG. 7B is a schematic cross-sectional view taken along the line B-B’ in FIG. 6.

As illustrated in FIGS. 6, 7A, and FIG. 7B, the sensor part 10A according to the first modification differs from the sensor part 10 illustrated in FIGS. 2, 3A, and 3B in that the substrate 110 has another cavity 112. Other basic configurations are the same as those of the sensor part 10 illustrated in FIGS. 2, 3A, and FIG. 3B , so the same reference numerals are given to the same elements, and overlapping description will be omitted.

In the area where the cavity 112 is formed, the insulating film 121 of the substrate 110 is removed, and the thickness of the main body 120 of the substrate 110 in the Z-direction is locally reduced, or both the main body 120 of the substrate 110 and insulating film 121 are removed. In the first modification, the thermistor Rd1 is disposed so as to overlap the cavity 112 in a plan view as seen from the Z-direction. In the example illustrated in FIG. 7B, both the main body 120 of the substrate 110 and insulating film 121 are removed in the area where the cavity 112 is formed, whereby the thermistor Rd1 is supported on the substrate 110 through the insulating films 122 and 123. The thickness of the substrate 110 in the Z-direction at the first portion 118 located between the cavities 111 and 112 is larger than that of portions of the substrate 110 that overlap either the cavity 111 or cavity 112. Heat from the heated heater MH1 is conducted to the thermistor Rd1 mainly through the first portion 118 of the substrate located between the cavities 111 and 112.

As in the sensor part 10A according to the first modification, when another cavity 112 is formed in the substrate 110 not only at the position overlapping the heater MH1 but also at the position overlapping the thermistor Rd1, the heat capacity in the vicinity of the thermistor Rd1 is reduced. As a result, the temperature responsiveness of the thermistor Rd1 to heating by the heater MH1 is enhanced, making it possible to measure CO₂ gas concentration in a shorter time. The temperature of the thermistor Rd1 when the heater MH1 is heated can be adjusted by the distance D2 between the cavities 111 and 112.

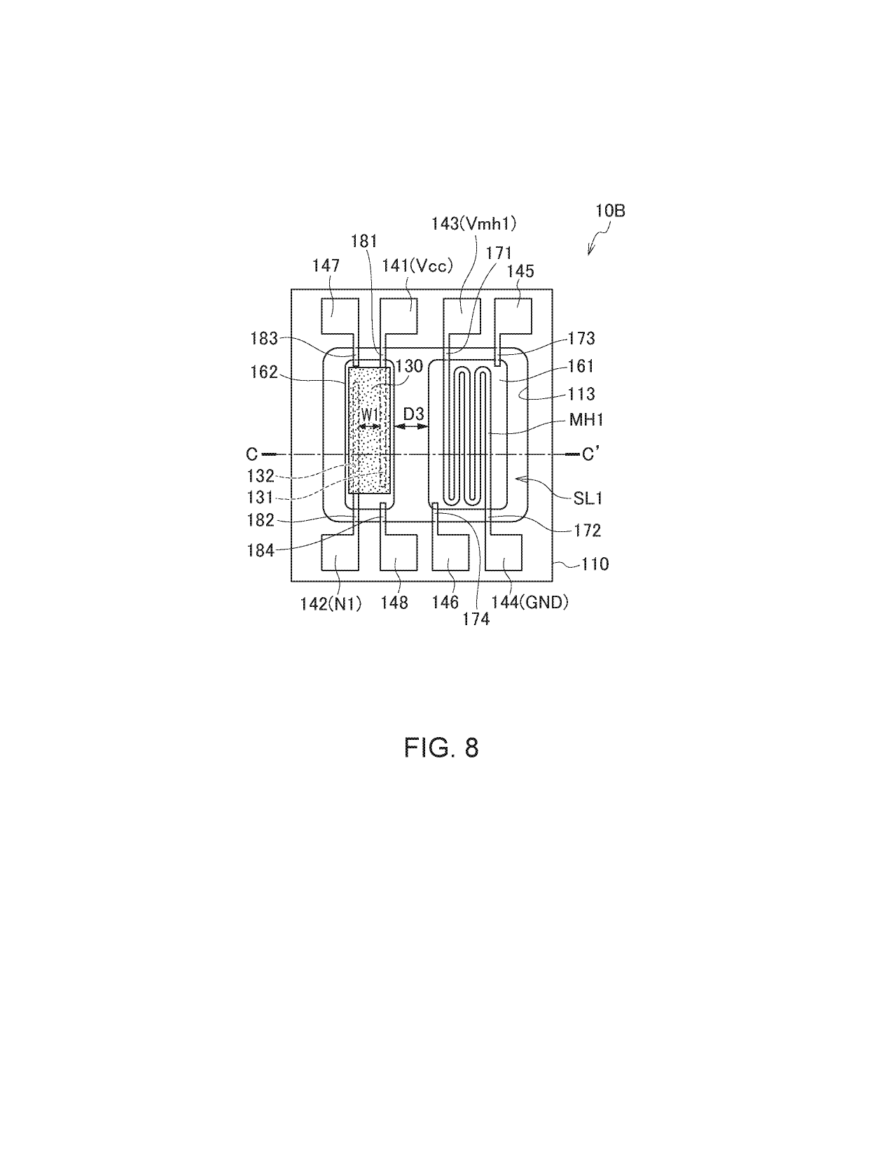

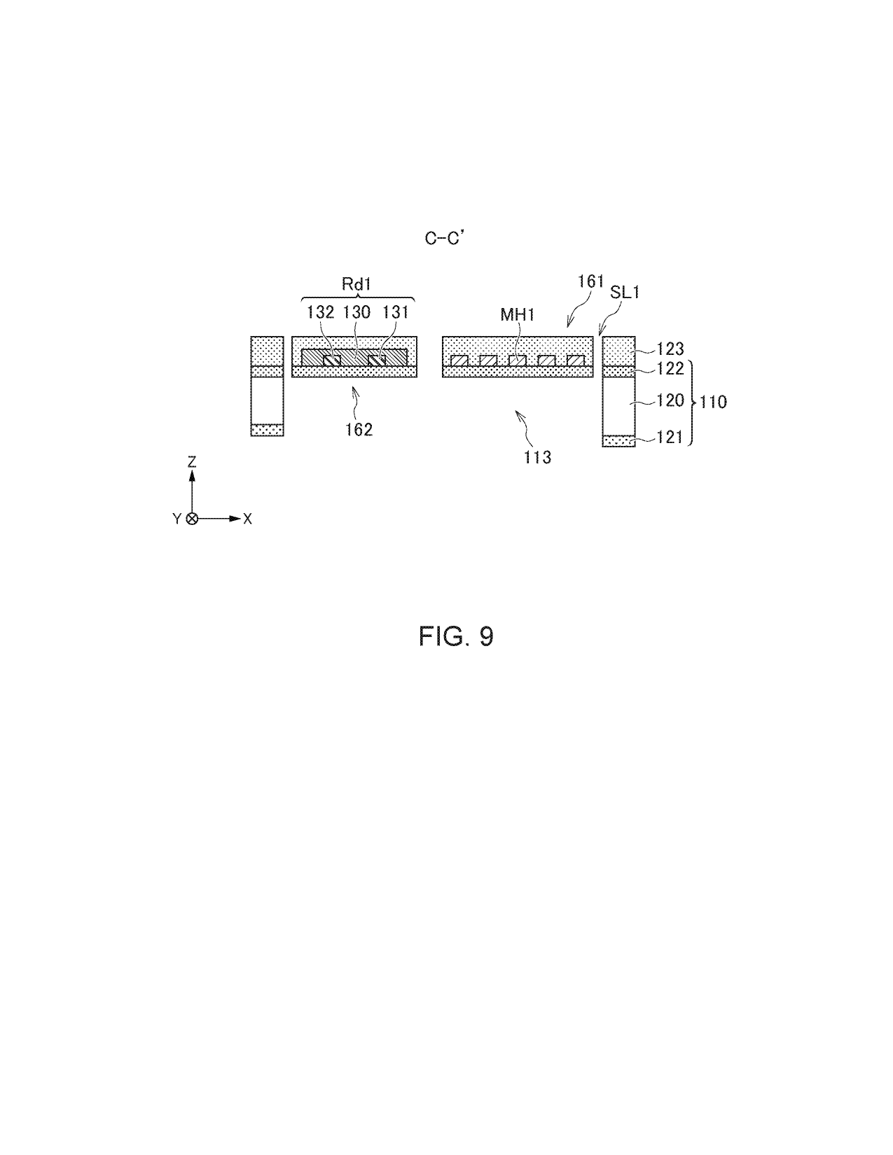

FIG. 8 is a schematic plan view illustrating the configuration of a sensor part 10B according to a second modification of the first embodiment. FIG. 9 is a schematic cross-sectional view taken along the line C-C’ in FIG. 8.

As illustrated in FIGS. 8 and 9, in the sensor part 10B according to the second modification, a cavity 113 is formed in the substrate 110, and the heater MH1 and the thermistor Rd1 are arranged so as to overlap the cavity 113. In a region overlapping the cavity 113, a slit SL1 is formed through the insulating films 122 and 123, except for the portions overlapping the heater MH1 and thermistor Rd1 and the portions surrounding them. As a result, the heater MH1 and the insulating films 122 and 123 supporting it constitute a membrane 161 overlapping the cavity 113, while the thermistor Rd1 and the insulating films 122 and 123 supporting it constitute a membrane 162 overlapping the cavity 113. In the present embodiment, the heater MH1 and the thermistor Rd1 are spaced apart from each other.

The membranes 161 and 162 may retain a portion of the main body 120 of the substrate 110, or the main body 120 of the substrate 110 and the insulating film 121 may be completely removed, with the insulating film 122 left exposed on the back side. In the example illustrated in FIG. 9, the main body 120 of the substrate 110 and the insulating film 121 are removed in the region where the cavity 113 is formed, thereby exposing the insulating film 122 from the back side of the membranes 161 and 162.

The membrane 161 is supported on the substrate 110 through bridges composed of wirings 171 and 172 connecting the heater MH1 to terminal electrodes 143 and 144, respectively, wirings 173 and 174 connected to dummy electrodes 145 and 146, respectively, and insulating films 122 and 123 located around these wirings 171 to 174. The membrane 162 is supported on the substrate 110 through bridges composed of a wiring 181 connecting the thermistor electrode 131 to the terminal electrode 141, a wiring 182 connecting the thermistor electrode 132 to the terminal electrode 142, wirings 183 and 184 connected to dummy electrodes 147 and 148, respectively, and insulating films 122 and 123 located around these wirings 181 to 184. The bridges supporting the membranes 161 and 162 may include a part of the substrate 110.

Other basic configurations of the sensor part 10B are the same as those of the sensor part 10 illustrated in FIGS. 2, 3A, and FIG. 3B, so the same reference numerals are given to the same elements, and overlapping description will be omitted.

As in the sensor part 10B according to the second modification, the heater MH1 and the thermistor Rd1 may be arranged in the same cavity 113. In this case, the heat generated by the heater MH1 is conducted to the thermistor Rd1 mainly through the bridges supporting the membrane 161, the substrate 110, and the bridges supporting the membrane 162. Thus, by arranging the heater MH1 and the thermistor Rd1 in the same cavity 113, the substrate 110 can be downsized. The temperature of the thermistor Rd1 when the heater MH1 is heated can be adjusted by the distance D3 between the membrane 161 and the membrane 162.

### <Second Embodiment>

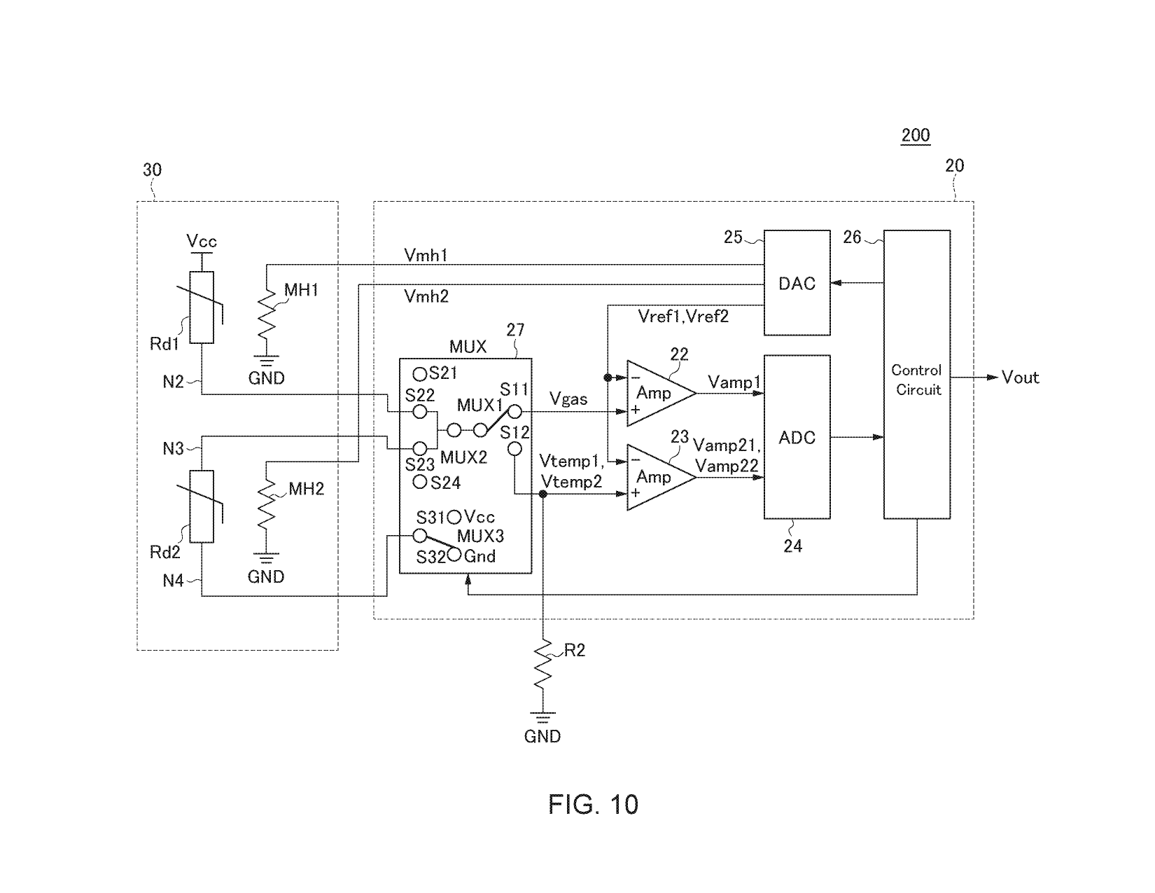

FIG. 10 is a circuit diagram illustrating the configuration of a gas sensor 200 according to a second embodiment of the technology described herein.

As illustrated in FIG. 10, the gas sensor 200 according to the second embodiment differs from the gas sensor 100 according to the first embodiment in the following points: the sensor part 10 is replaced with a sensor part 30; the multiplexer 21 included in the signal processing circuit 20 is replaced with a multiplexer 27; and a fixed resistor R2 is additionally provided. Other basic configurations are the same as those of the gas sensor 100 according to the first embodiment, so the same reference numerals are given to the same elements, and overlapping description will be omitted.

The sensor part 30 includes the thermistor Rd1 connected between the power supply Vcc and a node N2, the thermistor Rd2 connected between nodes N3 and N4, and the heaters MH1 and MH2. The thermistor Rd1 changes its temperature in accordance with variations in the temperature of the heater MH1. The thermistor Rd2 changes its temperature in accordance with variations in the temperature of the heater MH2. The thermistor Rd2 is a temperature-sensitive element whose resistance value varies with temperature.

During the gas detection period, the heater MH1 is heated to the first temperature region, while the second heater MH2 is heated to a second temperature range. The first temperature range refers to a predetermined range, for example, from 100°C to 230°C, or around 170°C. The second temperature range refers to a predetermined range, for example, from 250°C to 450°C, or around 340°C. The temperature range around 340°C may be from 339.5°C to 340.5°C, for example. The second temperature range is higher than the first temperature range, and the signal processing circuit 20 heats the heater MH2 to a temperature higher than that of the heater MH1 during the gas concentration measurement.

In the second temperature range, the ratio between the thermal conductivity of CO₂ gas and the thermal conductivity of air is closer to 1 than in the first temperature range. Thus, even when CO₂ gas is present in the measurement atmosphere in a state in which a fixed power is applied to the heater MH2 to heat it to the second temperature range, the change in the temperature of the heater MH2 due to the concentration of the CO₂ gas is small. Therefore, in the case where the heater MH2 is heated to 340°C under the condition that the CO₂ gas concentration in the measurement atmosphere is at the normal atmospheric level (e.g., 400 ppm), even when the CO₂ gas concentration in the measurement atmosphere exceeds the normal level, the temperature of the heater MH2 exhibits only a slight change in accordance with the concentration and is substantially maintained at 340°C. Thus, even when CO₂ gas is present in the measurement atmosphere in the state in which the heater MH2 is heated to the second temperature range, the change in the heat dissipation characteristics of the heater MH2 in accordance with the concentration is small, and accordingly, the change in the temperature of the thermistor Rd2, i.e., the change in the resistance value of the thermistor Rd2 in accordance with the concentration of CO₂ gas is also small.

The multiplexer 27 includes a first selection part MUX1, a second selection part MUX2, and a third selection part MUX3. The first selection part MUX1 is a circuit configured to connect one of the selection nodes S11 and S12 to the output node of the second selection part MUX2. The selection nodes S11 and S12 are connected to the differential amplifiers 22 and 23, respectively. The second selection part MUX2 is a circuit configured to short-circuit any two selected from the selection nodes S21 to S24 and connect them to the input node of the first selection part MUX1. The selection node S22 is connected to the thermistor Rd1 through the node N2. The selection node S23 is connected to the thermistor Rd2 through the node N3. The selection nodes S21 and S24 are dummy nodes and are in a floating state. The third selection part MUX3 is a circuit configured to connect one of the selection nodes S31 and S32 to the thermistor Rd2 through the node N4. The selection nodes S31 and S32 are connected to the power supply Vcc and the ground GND, respectively. The above-described selection operations of the multiplexer 27 are controlled by the control circuit 26.

The fixed resistor R2 is connected between the selection node S12 of the first selection part MUX1 and the ground GND.

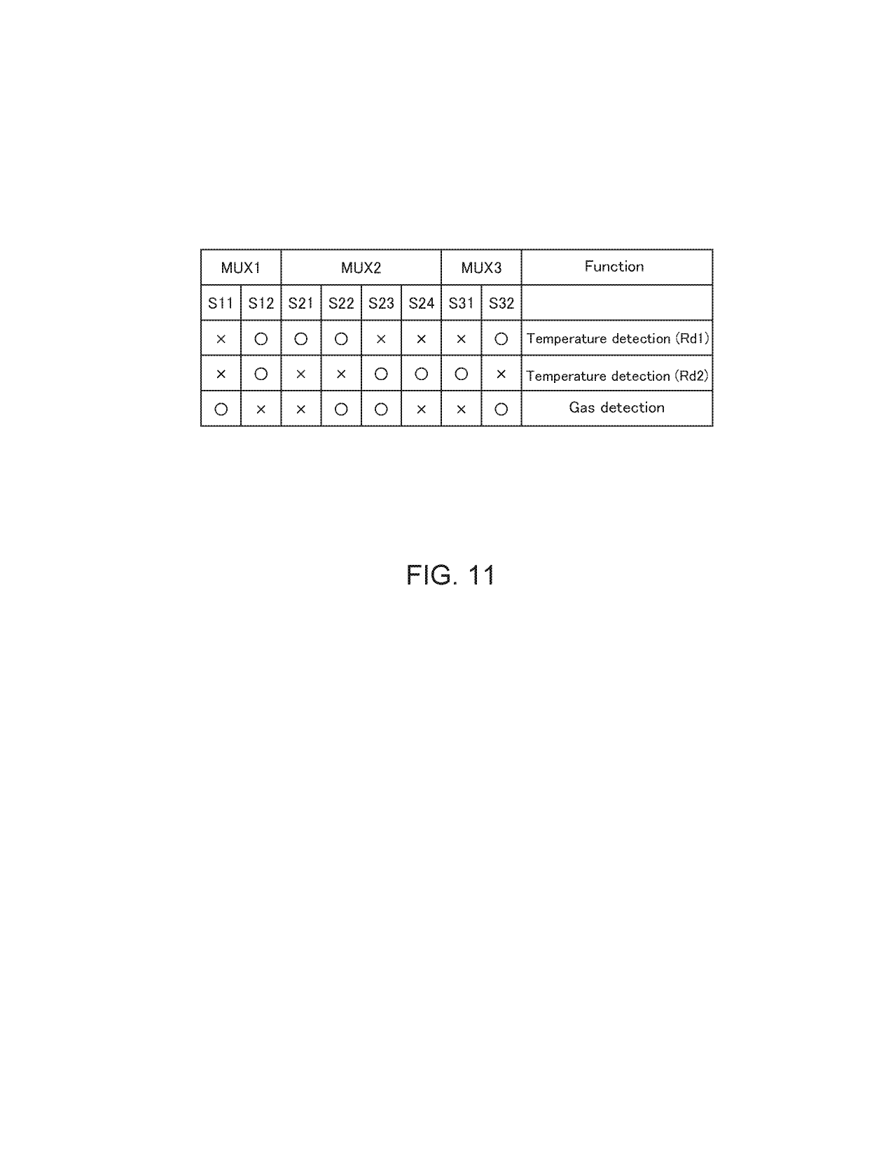

FIG. 11 is a table for explaining the function of the multiplexer 27. In FIG. 11, the circle mark (O) means “selected”, and the cross mark (×) indicates “not selected”.

When the ambient temperature is measured using the thermistor Rd1, the selection node S12 of the first selection part MUX1, the selection nodes S21 and S22 of the second selection part MUX2, and the selection node S32 of the third selection part MUX3 are selected. This connects the node N2 to the selection node S12. As a result, the thermistor Rd1 and the fixed resistor R2 are connected in series between the power supply Vcc and ground GND, and a temperature detection signal Vtemp1 appearing at the selection node S12 between the thermistor Rd1 and the fixed resistor R2 is supplied to the differential amplifier 23. The temperature detection signal Vtemp1 is an output value derived from the thermistor Rd1 indicating the ambient temperature. The differential amplifier 23 compares the temperature detection signal Vtemp1 with the reference potential Vref2 to generate an amplification signal Vamp21 corresponding to the amplified level difference (= Vtemp1 – Vref2) between the temperature detection signal Vtemp1 and the reference potential Vref2.

When the ambient temperature is measured using the thermistor Rd2, the selection node S12 of the first selection part MUX1, the selection nodes S23 and S24 of the second selection part MUX2, and the selection node S31 of the third selection part MUX3 are selected. This connects the node N3 to the selection node S12 and the node N4 to the power supply Vcc. As a result, the thermistor Rd2 and the fixed resistor R2 are connected in series between the power supply Vcc and ground GND, and a temperature detection signal Vtemp2 appearing at the selection node S12 between the thermistor Rd2 and the fixed resistor R2 is supplied to the differential amplifier 23. The temperature detection signal Vtemp2 is an output value derived from the thermistor Rd2 indicating the ambient temperature. The differential amplifier 23 compares the temperature detection signal Vtemp2 with the reference potential Vref2 to generate an amplification signal Vamp22 corresponding to the amplified level difference (= Vtemp2 – Vref2) between the temperature detection signal Vtemp2 and the reference potential Vref2.

The reference potential Vref2 used when the ambient temperature is measured using the thermistor Rd1 and the reference potential Vref2 used when the ambient temperature is measured using the thermistor Rd2 may have the same level or different levels.

When the gas concentration is measured, the selection node S11 of the first selection part MUX1, the selection nodes S22 and S23 of the second selection part MUX2, and the selection node S32 of the third selection part MUX3 are selected. As a result, the thermistors Rd1 and Rd2 are connected in series between the power supply Vcc and ground GND, and the gas detection signal Vgas appearing at the selection node S11 between the thermistors Rd1 and Rd2 is supplied to the differential amplifier 22.

The following describes the operation of the gas sensor 200 according to the second embodiment during the gas concentration measurement.

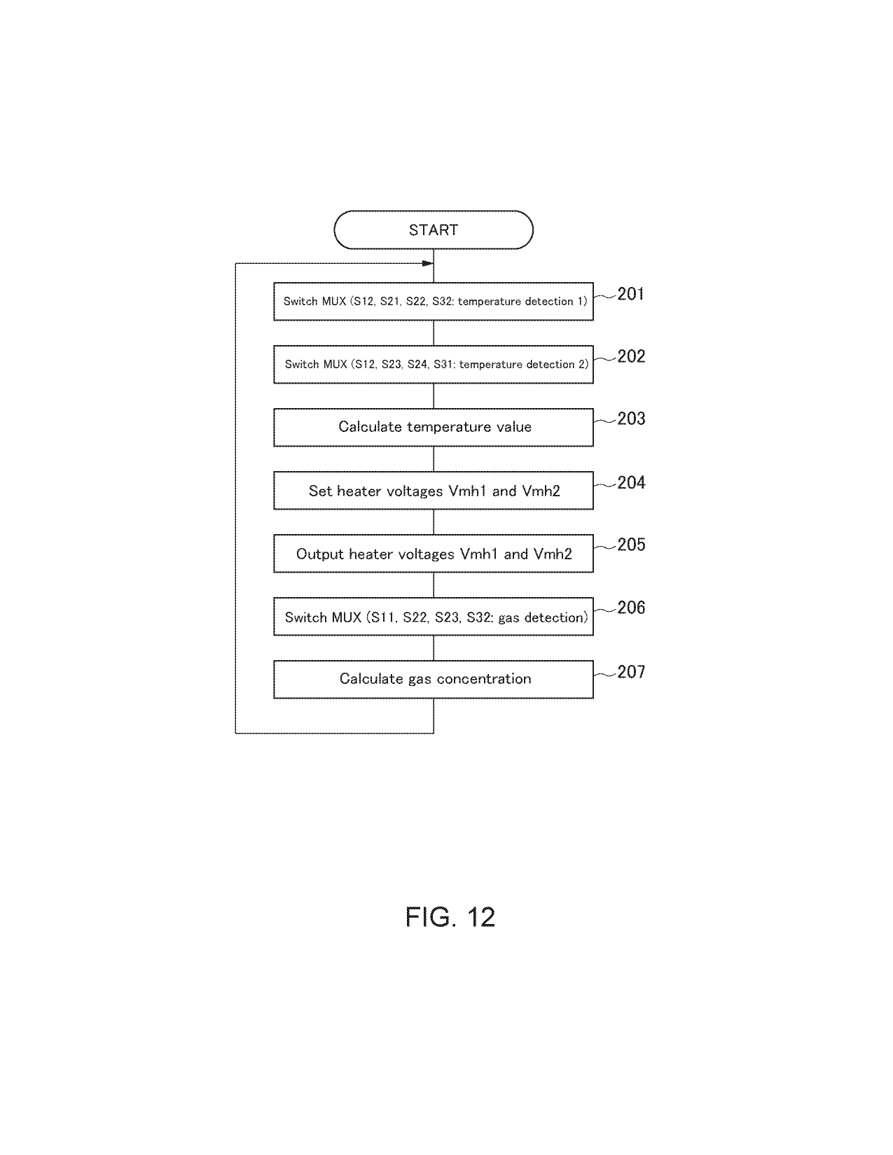

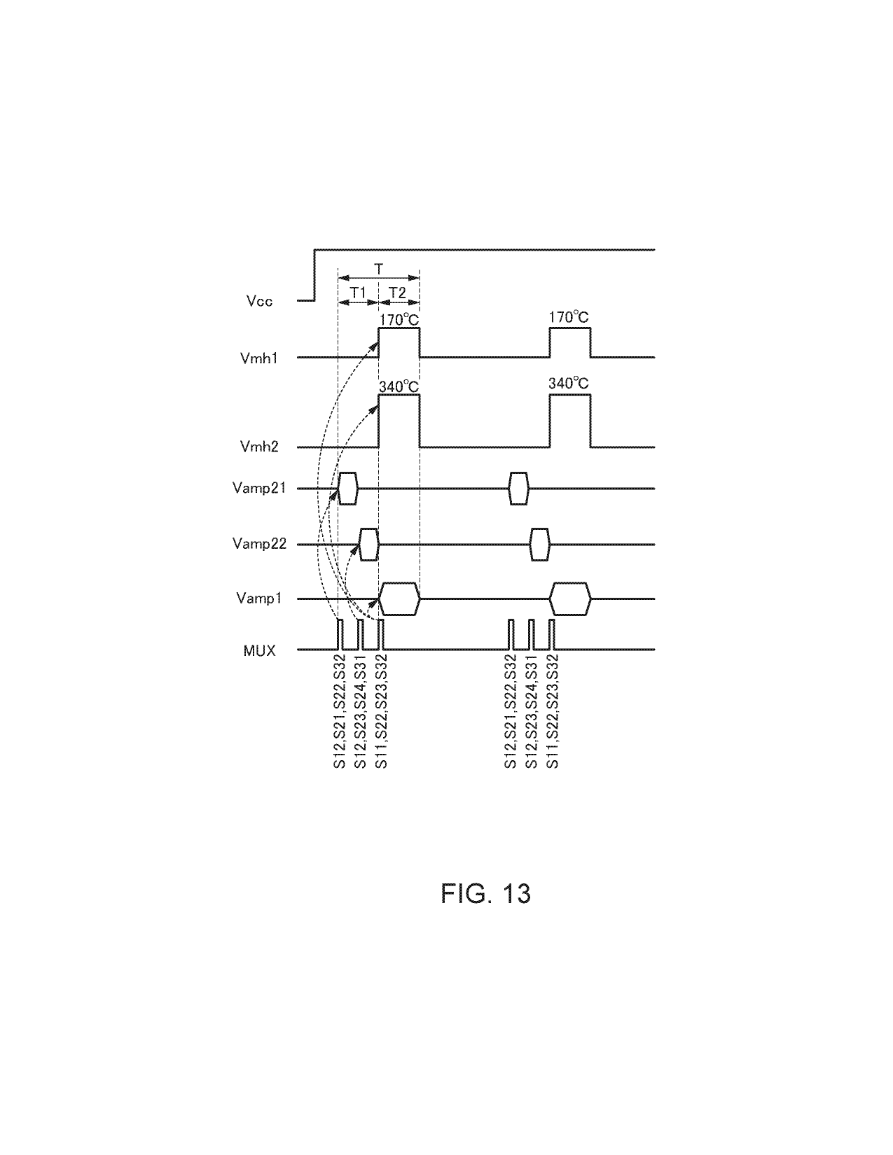

FIG. 12 is a flowchart for explaining the operation of the gas sensor 200 during the gas concentration measurement. FIG. 13 is a timing chart for explaining the operation of the gas sensor 200. The gas concentration measurement is executed in the period T illustrated in FIG. 13.

The control circuit 26 included in the signal processing circuit 20 controls the multiplexer 27 to select the selection nodes S12, S21, S22, and S23 and supplies the reference potential Vref2 to the differential amplifier 23 through the DA converter 25 (step 201). As a result, the amplification signal Vamp21 indicating the current ambient temperature that has been measured using the thermistor Rd1 is generated. At this time, the heater voltages Vmh1 and Vmh2 are not supplied to the heaters MH1 and MH2, respectively, so that the temperature of the thermistor Rd1 is maintained at the ambient temperature. Therefore, in this state, the temperature detection signal Vtemp1 appearing at the selection node S12 reflects the current ambient temperature. The temperature detection signal Vtemp1 is converted into the amplification signal Vamp21 by the differential amplifier 23 and thereafter converted into a corresponding digital value by the AD converter 24.

Then, the control circuit 26 included in the signal processing circuit 20 controls the multiplexer 27 to select the selection nodes S12, S23, S24, and S31 and supplies the reference potential Vref2 to the differential amplifier 23 through the DA converter 25 (step 202). As a result, the amplification signal Vamp22 indicating the current ambient temperature that has been measured using the thermistor Rd2 is generated. At this time, the heater voltages Vmh1 and Vmh2 are not supplied to the heaters MH1 and MH2, respectively, so that the temperature of the thermistor Rd2 is maintained at the ambient temperature. Therefore, in this state, the temperature detection signal Vtemp2 appearing at the selection node S12 reflects the current ambient temperature. The temperature detection signal Vtemp2 is converted into the amplification signal Vamp22 by the differential amplifier 23 and thereafter converted into a corresponding digital value by the AD converter 24.

Then, the control circuit 26 calculates the current ambient temperature based on the AD-converted amplification signals Vamp21 and Vamp22 (step 203). However, it is not essential to perform both step 201 and step 202, and the current ambient temperature may be calculated by performing only one of them, i.e., the current ambient temperature may be calculated based on only one of the amplification signals Vamp21 and Vamp22. This makes it possible to reduce the time required to measure the ambient temperature. These operations are executed in the temperature detection period T1 illustrated in FIG. 13. The temperature detection period T1 corresponds to the first half of the gas concentration measurement period T.

Then, the control circuit 26 calculates the heater voltage Vmh1 based on the ambient temperature that has been calculated based on the amplification signal Vamp21 and calculates the heater voltage Vmh2 based on the ambient temperature that has been calculated based on the amplification signal Vamp22 (step 204). For example, when the CO₂ gas concentration in the measurement atmosphere is at the normal atmospheric level (e.g., 400 ppm) irrespective of the current ambient temperature, the level of the heater voltage Vmh1 is set so as to heat the heater MH1 to 170°C and the level of the heater voltage Vmh2 is set so as to heat the heater MH2 to 340°C. Step 203 may be omitted, and in step 204, the heater voltages Vmh1 and Vmh2 may be calculated directly based on the amplification signals Vamp21 and Vamp22, respectively. The heater voltages Vmh1 and Vmh2 thus calculated are supplied to the heaters MH1 and MH2, respectively (step 205). By thus calculating the heater voltage Vmh1 to be applied to the heater MH1 disposed near the thermistor Rd1 based on the amplification signal Vamp21, which is an output value derived from the thermistor Rd1 and calculating the heater voltage Vmh2 to be applied to the heater MH2 disposed near the thermistor Rd2 based on the amplification signal Vamp22, which is an output value derived from the thermistor Rd2, the heating temperatures of heaters MH1 and MH2 can be controlled with higher accuracy. Alternatively, in step S204, the heater voltages Vmh1 and Vmh2 may be calculated based on only one of the amplification signals Vamp21 and Vamp22. Further alternatively, when the temperature calculated based on the amplification signal Vamp21 and the temperature calculated based on the amplification signal Vamp22 differ, the average value thereof may be regarded as the current ambient temperature.

In this state, the control circuit 26 controls the multiplexer 27 to select the selection nodes S11, S22, S23, and S32 and supplies the reference potential Vref1 to the differential amplifier 22 through the DA converter 25 (step 206). As a result, At the selection node S11, the gas detection signal Vgas having a level obtained by dividing the power supply VCC with the resistance value of the thermistor Rd1 and that of the thermistor Rd2 appears.

As described above, when CO₂ gas is present in the measurement atmosphere in a state in which the heater MH1 is heated to the first temperature range, the heat dissipation characteristics of the heater MH1 change in accordance with the concentration of the CO₂ gas. This change appears as a change in the temperature of the thermistor Rd1, i.e., a change in the resistance value thereof. On the other hand, even when CO₂ gas is present in the measurement atmosphere in a state in which the heater MH2 is heated to the second temperature range, the change in the heat dissipation characteristics of the heater MH2 in accordance with the concentration of the CO₂ gas is small. Accordingly, the change in the temperature of the thermistor Rd2, i.e., the change in the resistance value of the thermistor Rd2 in accordance with the concentration of CO₂ gas is also small. As a result, when the heaters MH1 and MH2 are heated to the first and second temperature ranges, respectively, the gas detection signal Vgas corresponding to the concentration of CO₂ gas in the measurement atmosphere appears at the selection node S11.

On the other hand, even when another gas whose heat dissipation characteristics exhibit no significant difference between when the heater MH1 is heated to the first temperature range and when the heater MH2 is heated to the second temperature range is contained in the measurement atmosphere, the concentration of this gas has little influence on the level of the gas detection signal Vgas. This allows the sensor part 30 to selectively detect the concentration of CO₂ gas.

The gas detection signal Vgas is converted into the amplification signal Vamp1 by the differential amplifier 22. The control circuit 26 calculates the concentration of CO₂ gas based on the AD-converted amplification signal Vamp1 and outputs the output signal Vout indicating the CO₂ gas concentration (step 207). The operations from steps 204 to 207 are executed in the gas detection period T2 illustrated in FIG. 13. The gas detection period T2 corresponds to the latter half of the gas concentration measurement period T.

Such periodic gas concentration measurement makes it possible to periodically detect a change in the concentration of CO₂ gas in the measurement atmosphere.

As described above, in the gas sensor 200 according to the present embodiment, the gas detection signal Vgas is obtained from the node between the series-connected thermistors Rd1 and Rd2, and the temperature of the thermistor Rd1 depends on the heating temperature of the heater MH1 changing in accordance with the concentration of CO₂ gas, while the temperature of the thermistor Rd2 depends on the heating temperature of the heater MH2 which exhibits only a slight change in accordance with the concentration of CO₂ gas. This makes it possible to selectively detect the concentration of CO₂ gas while reducing the influence of gases other than CO₂ gas.

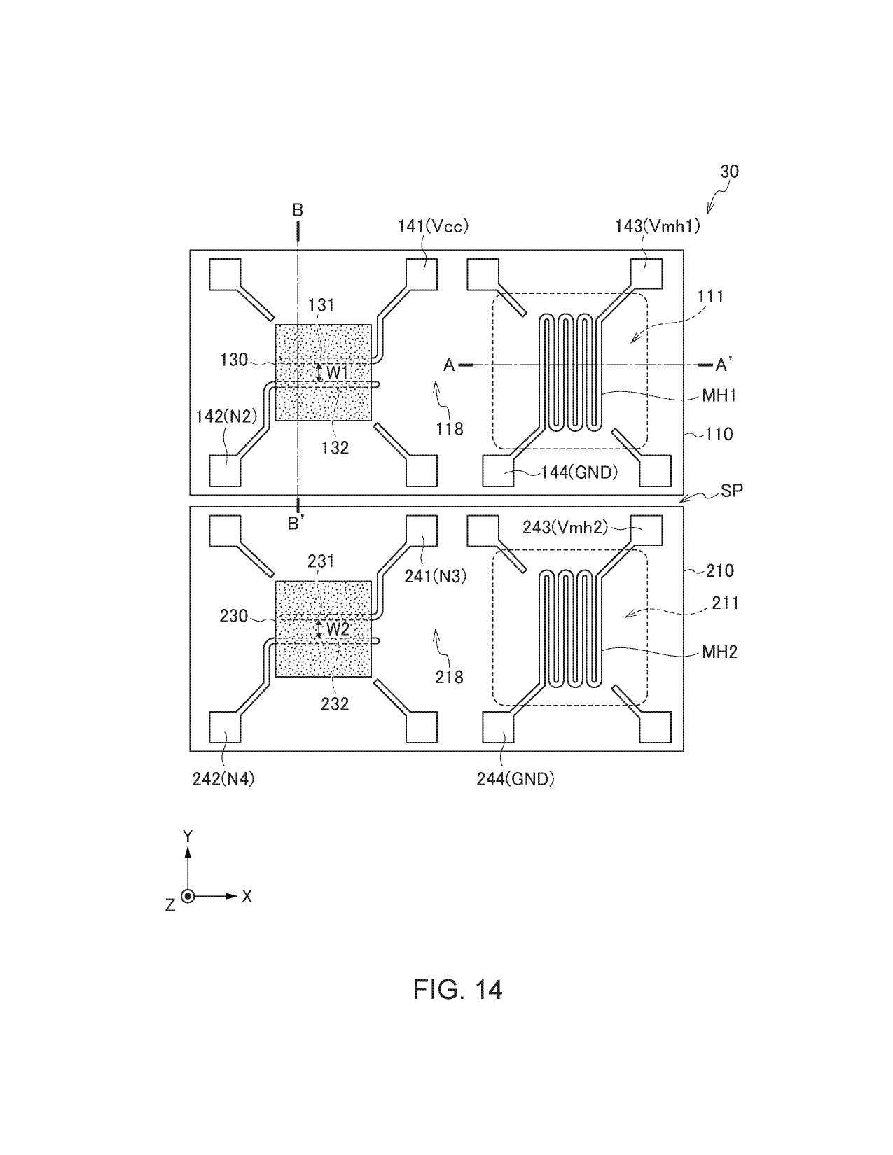

FIG. 14 is a schematic plan view illustrating the configuration of the sensor part 30. The cross sections taken along the line A-A’ and the line B-B’ in FIG. 14 are as illustrated in FIGS. 3A and 3B.

As illustrated in FIG. 14 the sensor part 30 includes two substrates 110 and 210. The substrate 110 supports thereon the heater MH1 and the thermistor Rd1, and the substrate 210 supports thereon the heater MH2 and the thermistor Rd2. The substrates 110 and 210 are separate members and are arranged so as to form a space SP therebetween. The configuration of the substrate 110, and the configurations of the heater MH1 and the thermistor Rd1, which are supported on the substrate 110, are as illustrated in FIG. 2.

The configuration of the substrate 210 is the same as that of the substrate 110. The configuration of the heater MH2 is the same as that of the heater MH1. The configuration of the thermistor Rd2 is the same as the thermistor Rd1. The configurations of the heater MH2 and the thermistor Rd2, which are supported on the substrate 210, are the same as those of the heater MH1 and the thermistor Rd1, which are supported on the substrate 110, respectively. That is, the substrate 210 has a cavity 211 at a position overlapping the heater MH2 in a plan view as seen from the Z-direction. One end of the heater MH2 is connected to a terminal electrode 243 that receives the heater voltage Vmh2, and the other end is connected to a terminal electrode 244 that receives the ground potential GND. The thermistor Rd2 includes a pair of thermistor electrodes 231 and 232 and a thermistor resistor 230 contacting the pair of thermistor electrodes 231 and 232. The thermistor electrode 231 is connected to the terminal electrode 241 constituting the node N3, and the thermistor electrode 232 is connected to the terminal electrode 242 constituting the node N4. The distance W2 between the thermistor electrodes 231 and 232 is, for example, 5 to 8 µm, and may be selected according to the target resistance value of the thermistor Rd2.

At the same temperature (e.g., room temperature), the ratio of the resistance value of the thermistor Rd2 measured between the thermistor electrodes 231 and 232 to the resistance value of the thermistor Rd1 measured between the thermistor electrodes 131 and 132 may be in the range of 0.9 to 1.1.

The thermistor Rd2 is disposed so as not to overlap the heater MH2. Thus, the thermistor Rd2 is not heated directly by the heater MH2, but is mainly heated by thermal conduction through the substrate 210. In the present embodiment, the heater MH2 and the thermistor Rd2 are spaced apart from each other with a portion (second portion 218) of the substrate 210 interposed therebetween. The thickness of the second portion 218 of the substrate 210 in the Z-direction is larger than that of a portion of the substrate 218 that overlaps the cavity 211 in the Z-direction. Further, in the present embodiment, the substrate 210 has no cavity at the position overlapping the thermistor Rd2.

Thus, in the present embodiment, the temperature of the thermistor Rd1 changes due to heat conducted from the heater MH1, mainly through the substrate 110, and in accordance with a change in the temperature of the heater MH1, and the temperature of the thermistor Rd2 changes due to heat conducted from the heater MH2, mainly through the substrate 210, and in accordance with a change in the temperature of the heater MH2. This significantly suppresses the temperature rise of the thermistors Rd1 and Rd2. For example, when the heater MH2 is heated from room temperature to 340°C, the temperature rise of the thermistor Rd2 is limited to only a few degrees above room temperature.

As described above, in the gas sensor 200 according to the present embodiment, the thermistor Rd1 and the heater MH1 are arranged at different locations on the substrate 110 so as not to overlap each other, and the thermistor Rd2 and the heater MH2 are arranged at different locations on the substrate 210 so as not to overlap each other. As a result, the temperature of the thermistor Rd1 during the gas concentration measurement is sufficiently lower than the heating temperature of the heater MH1, and the temperature of the thermistor Rd2 during the gas concentration measurement is sufficiently lower than the heating temperature of the heater MH2, making it possible to suppress aging of the thermistors Rd1 and Rd2 caused by exposure to high temperatures.

In addition, since the space SP is provided between the substrates 110 and 210, the heat of the heater MH1 is hardly conducted to the thermistor Rd2, and the heat of the heater MH2 is hardly conducted to the thermistor Rd1, thereby making it possible to reduce measurement errors due to thermal interference. The substrate 110 supporting thereon the heater MH1 and the thermistor Rd1 and the substrate 210 supporting thereon the heater MH2 and the thermistor Rd2 need not necessarily be separate members but may each be formed as a single members.

Further, when configured such that, at the same temperature, the ratio of the resistance value of the thermistor Rd2 measured between the electrodes to the resistance value of the thermistor Rd1 measured between the electrodes is in the range of 0.9 to 1.1, for example, the resistance value of the thermistor Rd1 between the electrodes and the resistance value of the thermistor Rd2 between electrodes become substantially equal, whereby the level of the gas detection signal Vgas can be made close to the level of Vcc/2, thereby enabling a wide dynamic range.

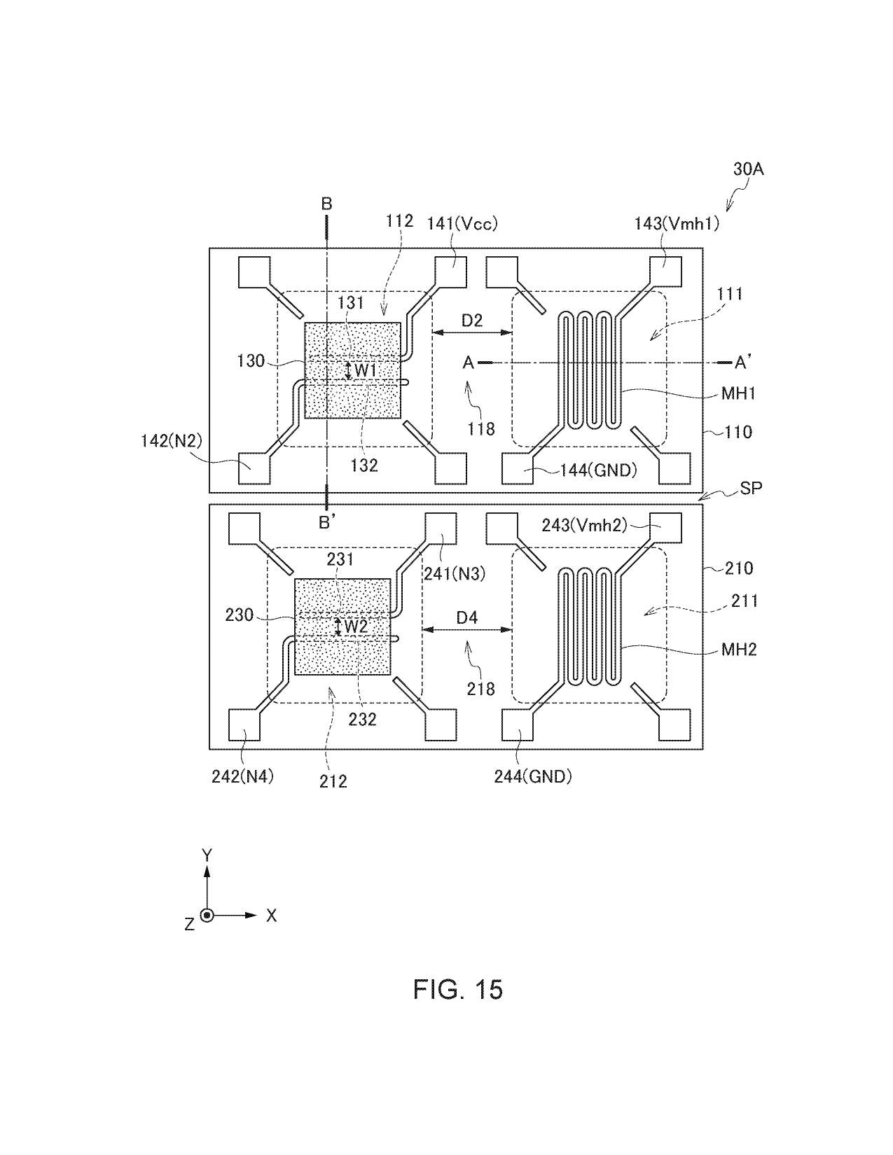

FIG. 15 is a schematic plan view illustrating the configuration of a sensor part 30A according to a first modification of the second embodiment. The cross sections taken along the line A-A’ and the line B-B’ in FIG. 15 are as illustrated in FIGS. 7A and 7B.

As illustrated in FIG. 15, the sensor part 30A according to the first modification differs from the sensor part 30 illustrated in FIG. 14 in that the substrate 110 has another cavity 112, and the substrate 210 has another cavity 212. Other basic configurations are the same as those of the sensor part 30 illustrated in 14, so the same reference numerals are given to the same elements, and overlapping description will be omitted.

In the area where the cavity 112 is formed, the insulating film 121 of the substrate 110 is removed, and the thickness of the main body 120 of the substrate 110 in the Z-direction is locally reduced, or both the main body 120 of the substrate 110 and insulating film 121 are removed. Similarly, in the area where the cavity 212 is formed, the thickness of the substrate 210 in the Z-direction is locally reduced. In the first modification, in a plan view as seen from the Z-direction, the thermistor Rd1 is disposed so as to overlap the cavity 112, and the thermistor Rd2 is disposed so as to overlap the cavity 212. The thickness of the substrate 110 in the Z-direction at the first portion 118 located between the cavities 111 and 112 is larger than that of portions of the substrate 110 that overlap either the cavity 111 or cavity 112. Similarly, the thickness of the substrate 210 in the Z-direction at the second portion 218 located between the cavities 211 and 212 is larger than that of portions of the substrate 210 that overlap either the cavity 211 or cavity 212.

As in the sensor part 30A according to the first modification, when another cavity 112 is formed in the substrate 110 not only at the position overlapping the heater MH1 but also at the position overlapping the thermistor Rd1, and another cavity 212 is formed in the substrate 210 not only at the position overlapping the heater MH2 but also at the position overlapping the thermistor Rd2, the heat capacity in the vicinity of the thermistors Rd1 and Rd2 is reduced. As a result, the temperature responsiveness of the thermistor Rd1 to heating by the heater MH1 is enhanced, and the temperature responsiveness of the thermistor Rd2 to heating by the heater MH2, making it possible to measure CO₂ gas concentration in a shorter time. The temperature of the thermistor Rd1 when the heater MH1 is heated can be adjusted by the distance D2 between the cavities 111 and 112, and the temperature of the thermistor Rd2 when the heater MH2 is heated can be adjusted by the distance D4 between the cavities 211 and 212.

The distance D4 may be larger than the distance D2. This makes the distance between the heater MH2 and the thermistor Rd2 larger than the distance between the heater MH1 and the thermistor Rd1 to reduce the difference in temperature between the thermistor Rd1 measured when the heater MH1 is heated to the first temperature range and the thermistor Rd2 measured when the heater MH2 is heated to the second temperature range. As a result, when configured such that, at the same temperature, the ratio of the resistance value of the thermistor Rd2 measured between the electrodes to the resistance value of the thermistor Rd1 measured between the electrodes is in the range of 0.9 to 1.1, for example, the level of the gas detection signal Vgas can be made closer to the level of Vcc/2, thereby enabling a wider dynamic range.

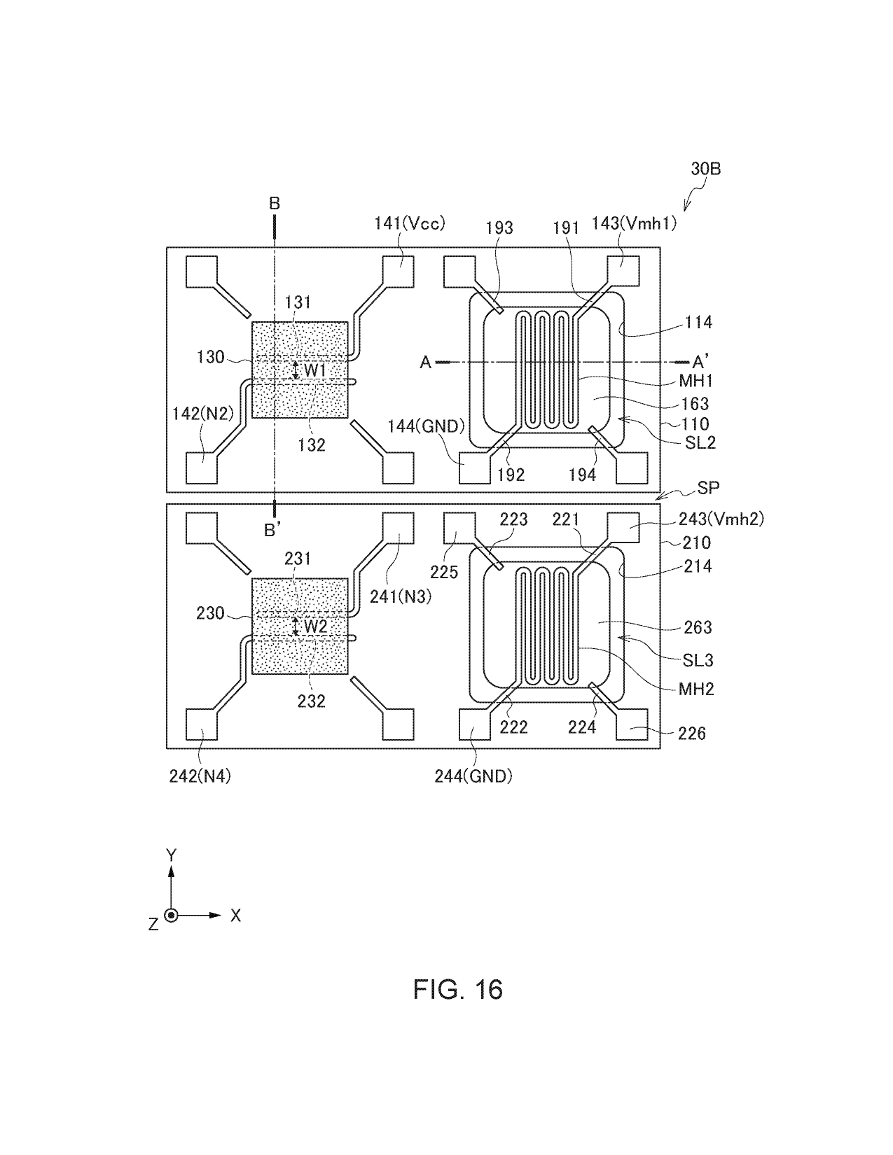

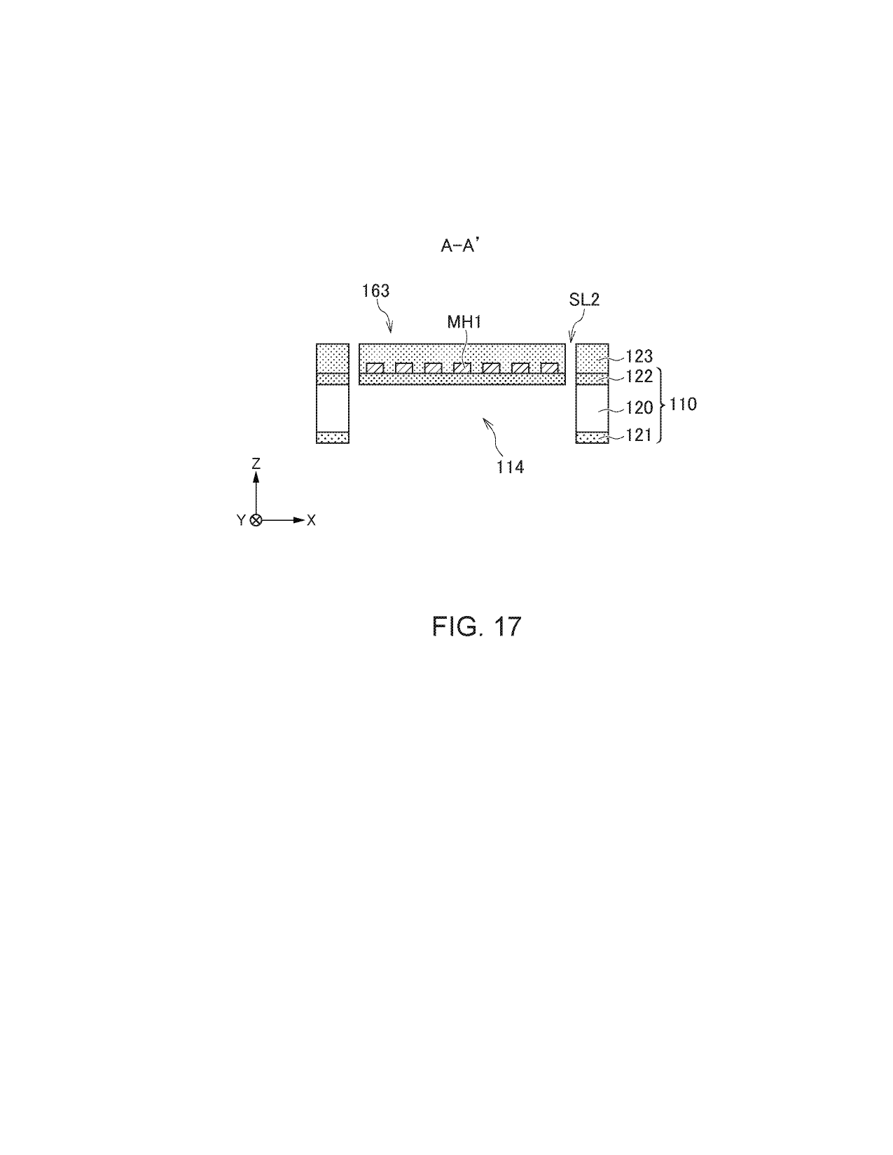

FIG. 16 is a schematic plan view illustrating the configuration of a sensor part 30B according to a second modification of the second embodiment. FIG. 17 is a schematic cross-sectional view taken along the line A-A’ in FIG. 16.

As illustrated in FIGS. 16 and 17, in the sensor part 30B according to the second modification, a cavity 114 and a cavity 214 are formed in the substrate 110 and the substrate 210, respectively, and the heaters MH1 and MH2 are disposed so as to overlap the cavities 114 and 214, respectively. A slit SL2 is formed in a region overlapping the cavity 114, and the heater MH1 is supported by a membrane 163. Similarly, a slit SL3 is formed in a region overlapping the cavity 214, and the heater MH2 is supported by a membrane 263.

The membrane 163 is supported on the substrate 110 through bridges composed of wirings 191 and 192 connecting the heater MH1 to terminal electrodes 143 and 144, respectively, wirings 193 and 194 connected to dummy electrodes 145 and 146, respectively, and insulating films located around these wirings 191 to 194. The bridges supporting the membrane 163 may include a portion of the substrate 110. The membrane 263 is supported on the substrate 210 through bridges composed of wirings 221 and 222 connecting the heater MH2 to terminal electrodes 243 and 244, respectively, wirings 223 and 224 connected to dummy electrodes 225 and 226, respectively, and insulating films located around these wirings 221 to 224. The bridges supporting the membrane 263 may include a portion of the substrate 210.

Other basic configurations of the sensor part 30B are the same as those of the sensor part 30 illustrated in FIG. 14, so the same reference numerals are given to the same elements, and overlapping description will be omitted.

As in the sensor part 30B according to the second modification, the heaters MH1 and MH2 may be disposed on the membranes 163 and 263, respectively.

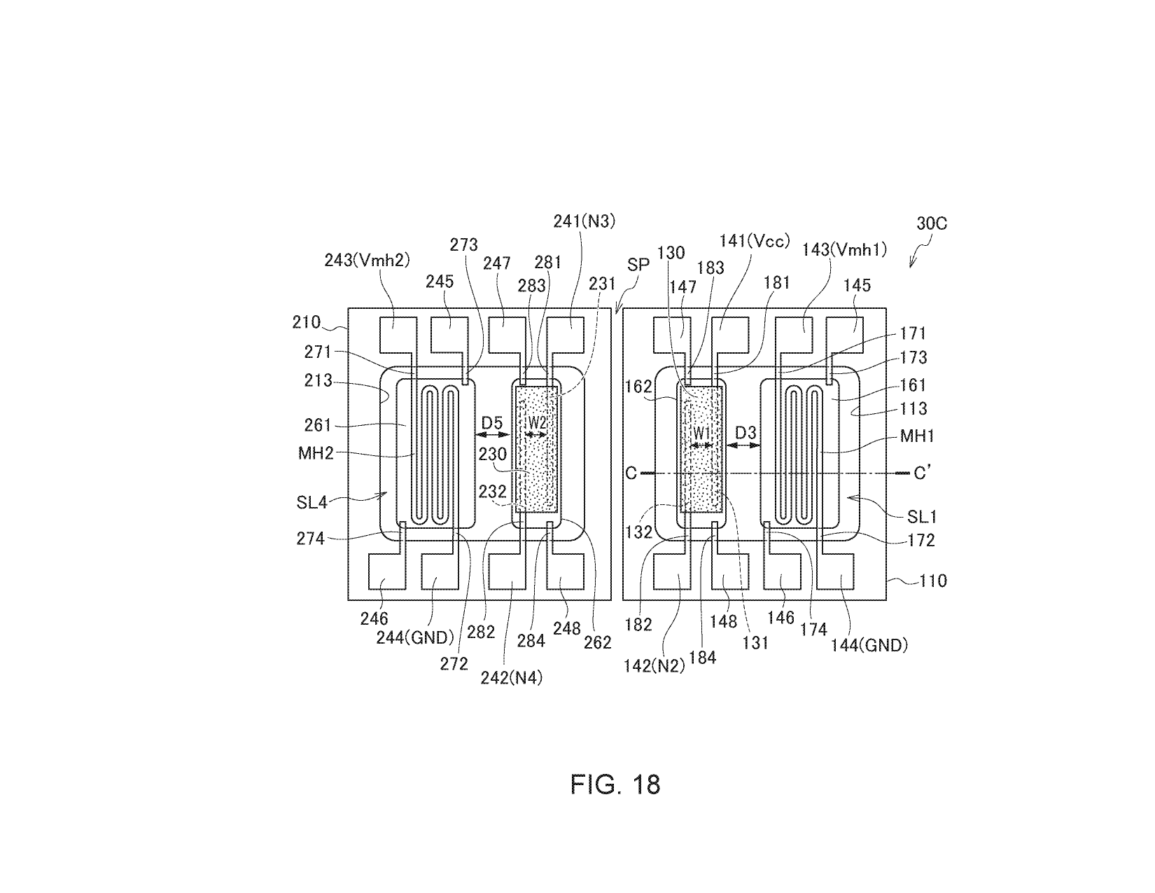

FIG. 18 is a schematic plan view illustrating the configuration of a sensor part 30C according to a third modification of the second embodiment. The cross sections taken along the line C-C’ in FIG. 18 are as illustrated in FIG. 9.

As illustrated in FIG. 18, in the sensor part 30C according to the third modification, the cavity 113 and a cavity 213 are formed in the substrate 110 and the substrate 210, respectively, and the heaters MH1 and MH2 are disposed so as to overlap the cavities 113 and 213, respectively. The slit SL1 is formed in a region overlapping the cavity 113, and the heater MH1 and the thermistor Rd1 are supported by the membranes 161 and 162, respectively. Similarly, a slit SL4 is formed in a region overlapping the cavity 213, and the heater MH2 and the thermistor Rd2 are supported by membranes 261 and 262, respectively. In the present modification, the heater MH1 and the thermistor Rd1 are spaced apart from each other, and the heater MH2 and the thermistor Rd2 are spaced apart from each other.

The membrane 261 is supported on the substrate 210 through bridges composed of wirings 271 and 272 connecting the heater MH2 to the terminal electrodes 243 and 244, respectively, wirings 273 and 274 connected to dummy electrodes 245 and 246, respectively, and insulating films located around these wirings 271 to 274. The membrane 262 is supported on the substrate 210 through bridges composed of a wiring 281 connecting the thermistor electrode 231 to the terminal electrode 241, a wiring 282 connecting the thermistor electrode 232 to the terminal electrode 242, wirings 283 and 284 connected to dummy electrodes 247 and 248, respectively, and insulating films located around these wirings 281 to 284. The bridges supporting the membranes 261 and 262 may include a portion of the substrate 210.

Other basic configurations of the sensor part 30C are the same as those of the sensor part 10 illustrated in FIG. 8 and the sensor part 30 illustrated in FIG. 14, so the same reference numerals are given to the same elements, and overlapping description will be omitted.

As in the sensor part 30C according to the third modification, the heater MH1 and the thermistor Rd1 may be arranged in the same cavity 113, and the heater MH2 and the thermistor Rd2 may be arranged in the same cavity 213. The temperature of the thermistor Rd2 when the heater MH2 is heated can be adjusted by the distance D5 between the membrane 261 and the membrane 262.

### <Third Embodiment>

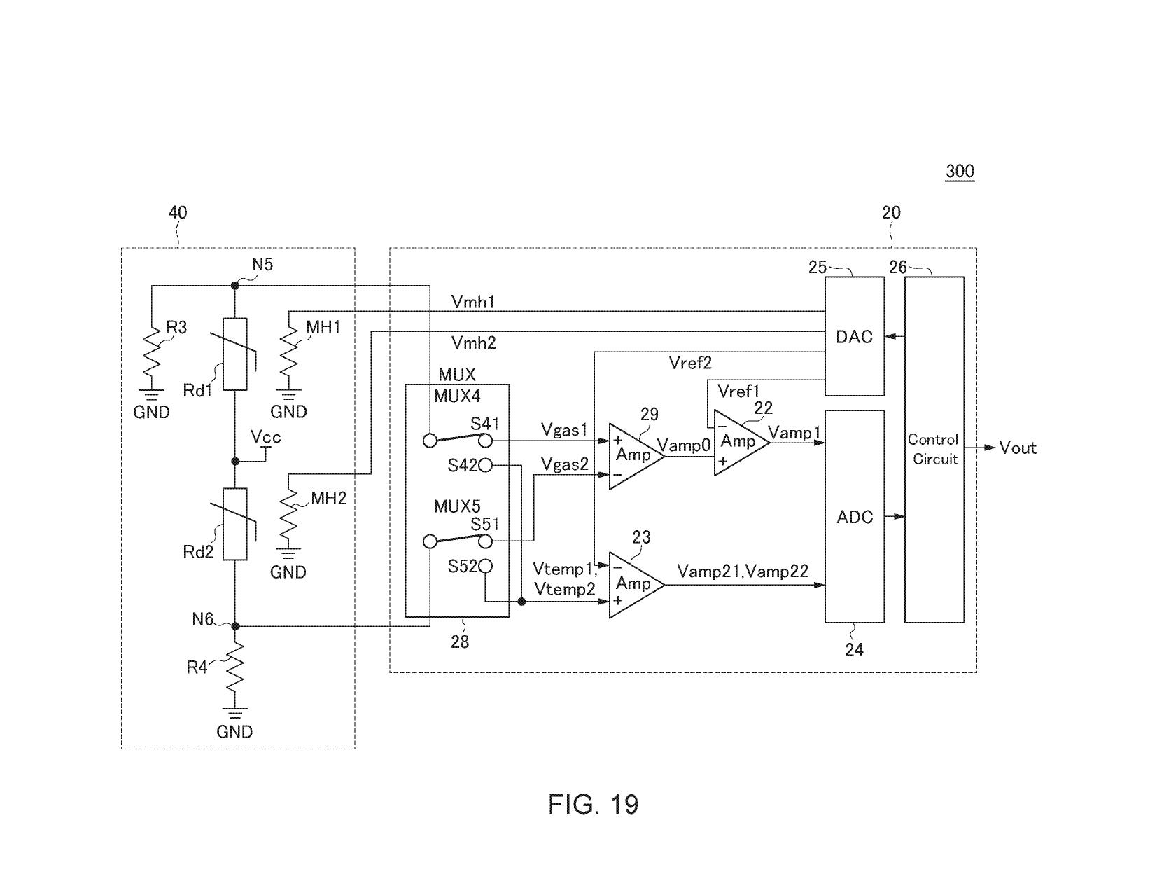

FIG. 19 is a circuit diagram illustrating the configuration of a gas sensor 300 according to a third embodiment of the technology described herein.

As illustrated in FIG. 19, the gas sensor 300 according to the third embodiment differs from the gas sensor 200 according to the second embodiment in the following points: the sensor part 30 is replaced with a sensor part 40; the multiplexer 27 included in the signal processing circuit 20 is replaced with a multiplexer 28; a differential amplifier 29 is provided between the multiplexer 28 and the differential amplifier 22; and the fixed resistor R2 is omitted. Other basic configurations are the same as those of the gas sensor 200 according to the second embodiment, so the same reference numerals are given to the same elements, and overlapping description will be omitted.

The sensor part 40 includes the thermistor Rd1 and a fixed resistor R3, which are connected in series in this order between the power supply Vcc and the ground GND, the thermistor Rd2 and a fixed resistor R4, which are connected in series in this order between the power supply Vcc and the ground GND, and the heaters MH1 and MH2. The thermistor Rd1 changes its temperature in accordance with variations in the temperature of the heater MH1, and the thermistor Rd2 changes its temperature in accordance with variations in the temperature of the heater MH2.

The multiplexer 28 includes a fourth selection part MUX4 and a fifth selection part MUX5. The fourth selection part MUX4 is a circuit configured to connect one of the selection nodes S41 and S42 to a node N5 between the thermistor Rd1 and the fixed resistor R3. A gas detection signal Vgas1 appears at the selection node S41, and the temperature detection signal Vtemp1 appears at the selection node S42. The fifth selection part MUX5 is a circuit configured to connect one of the selection nodes S51 and S52 to a node N6 between the thermistor Rd2 and the fixed resistor R4. A gas detection signal Vgas2 appears at the selection node S51, and the temperature detection signal Vtemp2 appears at the selection node S52. The above-described selection operations of the multiplexer 28 are controlled by the control circuit 26.

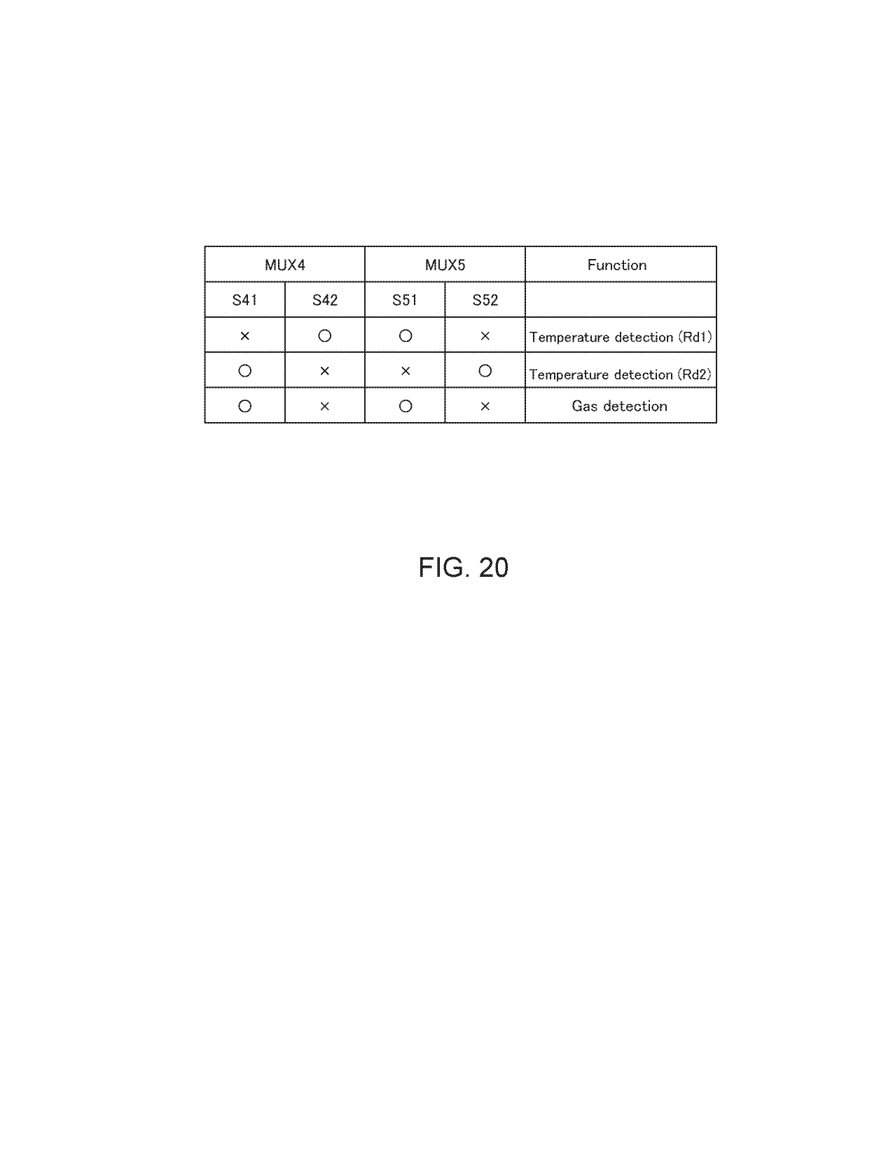

FIG. 20 is a table for explaining the function of the multiplexer 28. In FIG. 20, the circle mark (O) means “selected”, and the cross mark (×) indicates “not selected”.

When the ambient temperature is measured using the thermistor Rd1, the selection node S42 of the fourth selection part MUX4, and the selection node S51 of the fifth selection part MUX5 are selected. As a result, the temperature detection signal Vtemp1 appearing at the node N5 is supplied to the differential amplifier 23. The differential amplifier 23 compares the temperature detection signal Vtemp1 with the reference potential Vref2 to generate the amplification signal Vamp21 corresponding to the amplified level difference (= Vtemp1 – Vref2) between the temperature detection signal Vtemp1 and the reference potential Vref2.

When the ambient temperature is measured using the thermistor Rd2, the selection node S41 of the fourth selection part MUX4, and the selection node S52 of the fifth selection part MUX5 are selected. As a result, the temperature detection signal Vtemp2 appearing at the node N6 is supplied to the differential amplifier 23. The differential amplifier 23 compares the temperature detection signal Vtemp2 with the reference potential Vref2 to generate the amplification signal Vamp22 corresponding to the amplified level difference (= Vtemp2 – Vref2) between the temperature detection signal Vtemp2 and the reference potential Vref2.

When the gas concentration is measured, the selection node S41 of the fourth selection part MUX4, and the selection node S51 of the fifth selection part MUX5 are selected. As a result, the gas detection signal Vgas1 appearing at the node N5 and the gas detection signal Vgas2 appearing at the node N6 are supplied to the differential amplifier 29. The differential amplifier 29 compares the gas detection signal Vgas1 with the gas detection signal Vgas2 to generate an amplification signal Vamp0 corresponding to the amplified level difference (= Vgas1 – Vgas2) between the gas detection signal Vgas1 and the gas detection signal Vgas2. The amplification signal Vamp0 is supplied to the differential amplifier 22. The differential amplifier 22 compares the amplification signal Vamp0 with the reference potential Vref1 to generate the amplification signal Vamp1 corresponding to the amplified level difference (= Vamp0 – Vref1) between the amplification signal Vamp0 and the reference potential Vref1.

The mechanical configuration of the sensor part 40 may be the same as that of the sensor part 30 illustrated in FIG. 14, the sensor part 30A illustrated in FIG. 15, the sensor part 30B illustrated in FIGS. 16 and 17, or the sensor part 30C illustrated in FIG. 18.

As exemplified by the gas sensor 300 according to the third embodiment, the thermistors Rd1 and Rd2 need not be connected in series, but they may be connected in parallel between the power supply Vcc and the ground GND so as to calculate the concentration of a gas to be measured based on the difference between an output voltage (gas detection signal Vgas1) derived from the thermistor Rd1 and an output voltage (gas detection signal Vgas2) derived from the thermistor Rd2.

### <Fourth Embodiment>

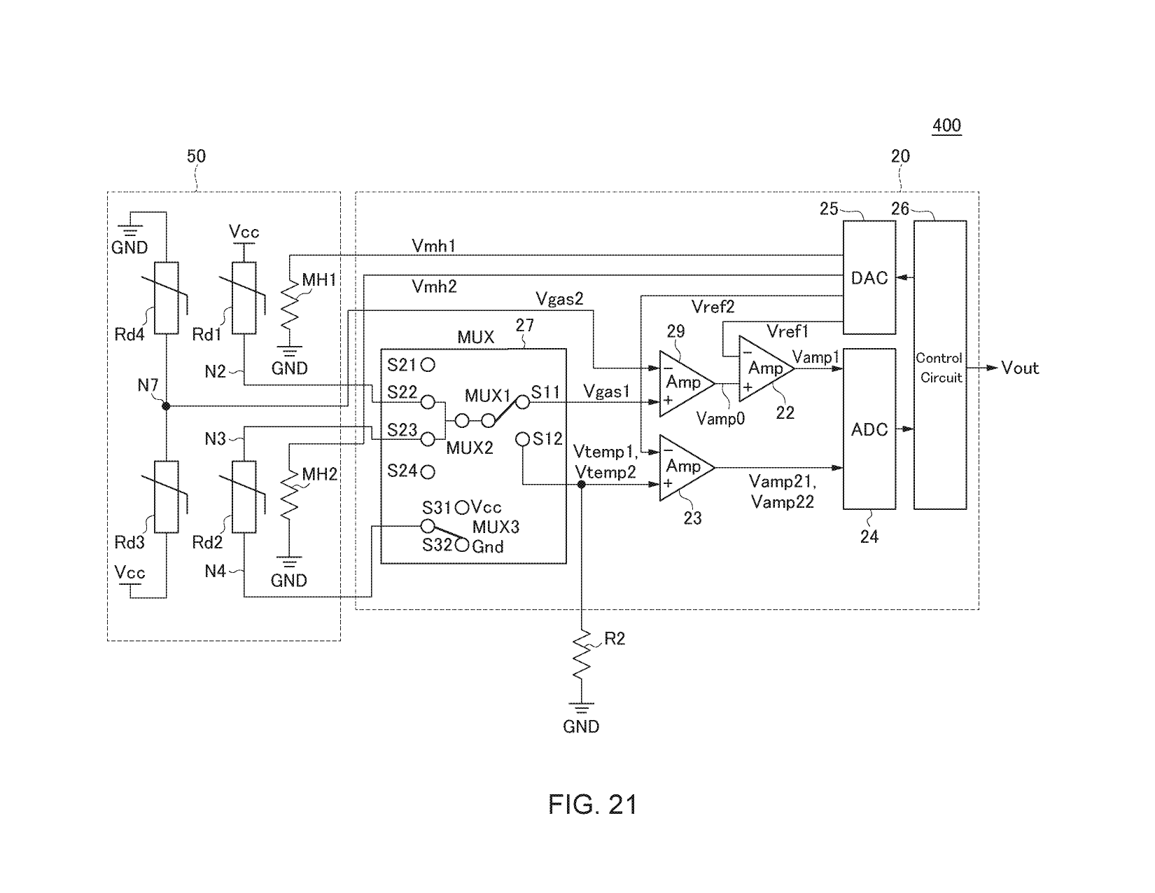

FIG. 21 is a circuit diagram illustrating the configuration of a gas sensor 400 according to a fourth embodiment of the technology described herein.

As illustrated in FIG. 21, the gas sensor 400 according to the fourth embodiment differs from the gas sensor 200 according to the second embodiment in that the sensor part 30 is replaced with a sensor part 50, and a differential amplifier 29 is provided between the multiplexer 27 and the differential amplifier 22. Other basic configurations are the same as those of the gas sensor 200 according to the second embodiment, so the same reference numerals are given to the same elements, and overlapping description will be omitted.

The sensor part 50 further includes a thermistor Rd3 and a thermistor Rd4, which are connected in series in this order between the power supply Vcc and the ground GND. The thermistors Rd3 and Rd4 are temperature-sensitive elements whose resistance value varies with temperature. The thermistor Rd4 changes its temperature in accordance with variations in the temperature of the heater MH1, and the thermistor Rd3 changes its temperature in accordance with variations in the temperature of the heater MH2. The node between the thermistors Rd4 and Rd3 is a node N7. The gas detection signal Vgas2 appears at the node N7. The gas detection signal Vgas1 appears at the selection node S11 of the first selection part MUX1.

The differential amplifier 29 compares the gas detection signal Vgas1 output from the multiplexer 27 with the gas detection signal Vgas2 appearing at the node N7 to generate the amplification signal Vamp0 corresponding to the amplified level difference (= Vgas1 – Vgas2) between the gas detection signals Vgas1 and Vgas2. The amplification signal Vamp0 is supplied to the differential amplifier 22. The differential amplifier 22 compares the amplification signal Vamp0 with the reference potential Vref1 to generate the amplification signal Vamp1 corresponding to the amplified level difference (= Vamp0 – Vref1) between the amplification signal Vamp0 and the reference potential Vref1.

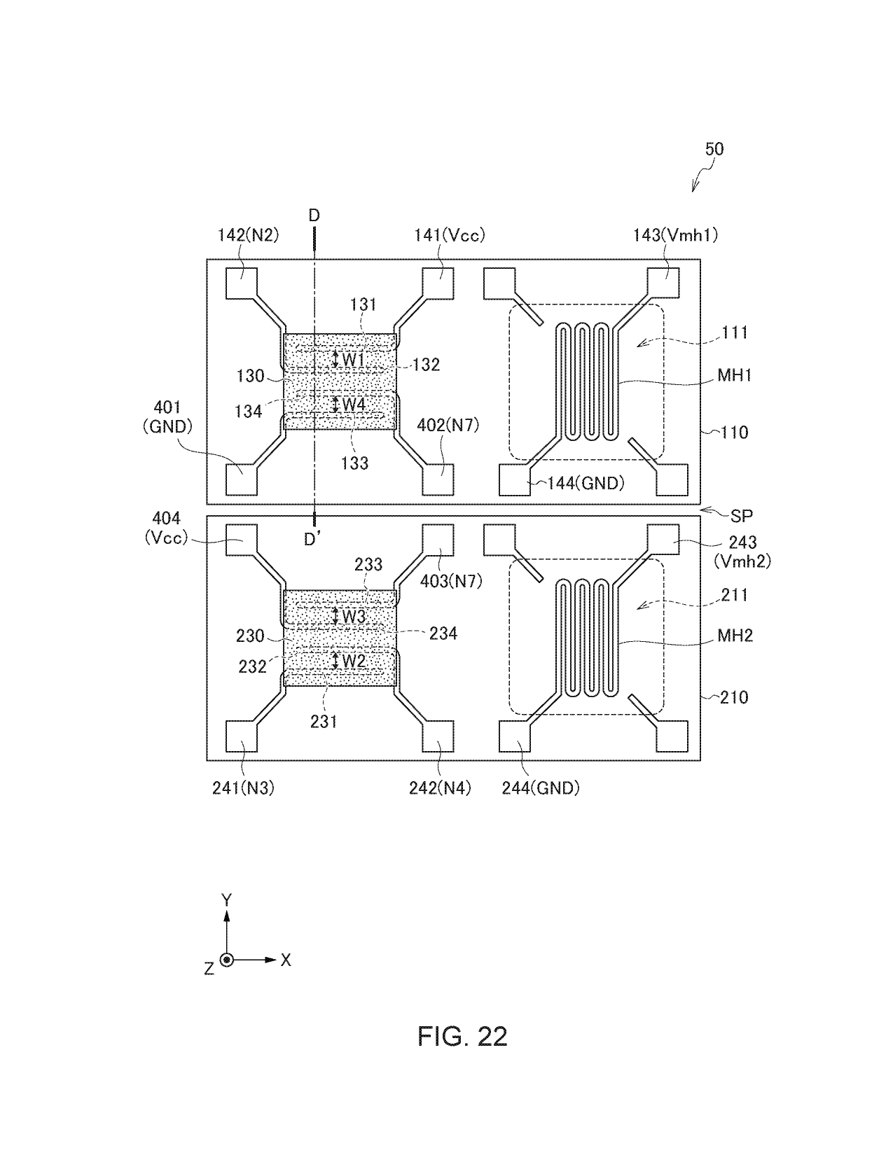

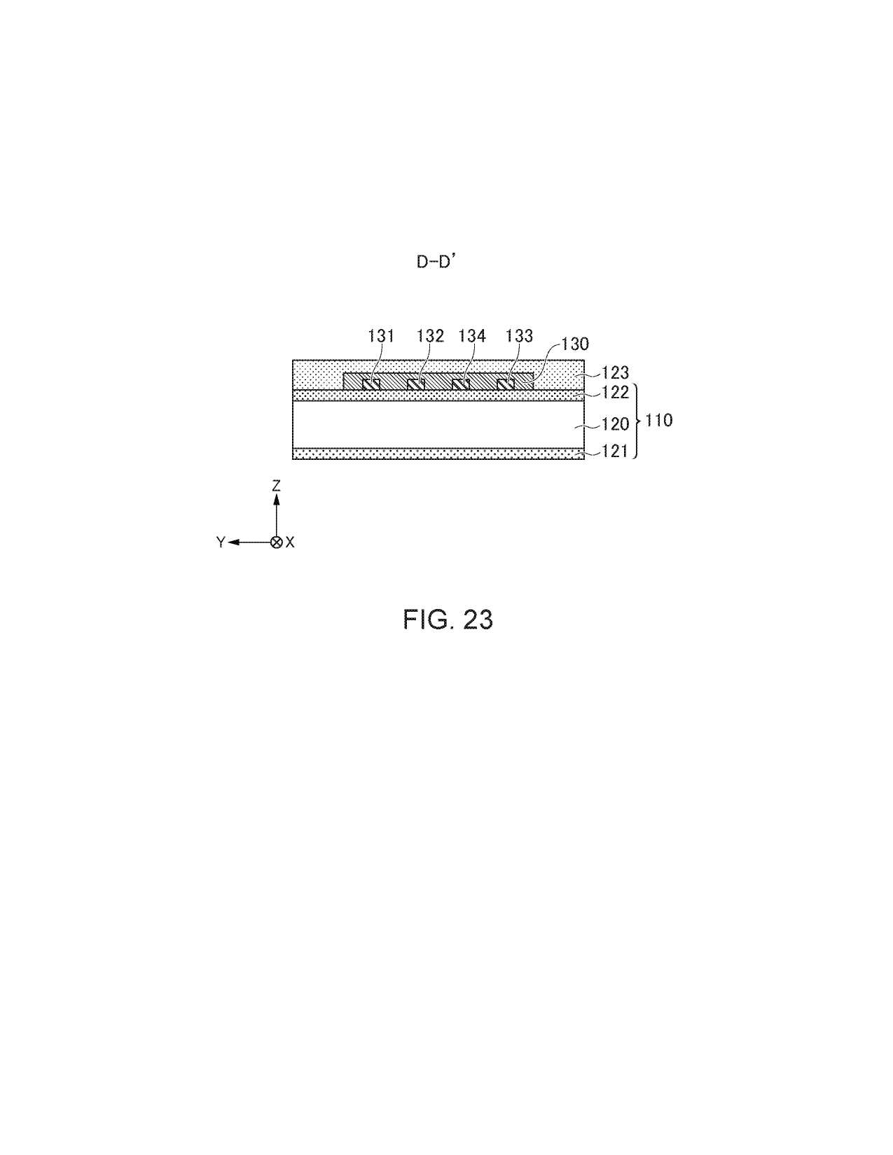

FIG. 22 is a schematic plan view illustrating the configuration of the sensor part 50. FIG. 23 is a schematic cross-sectional view taken along the line D-D’ in FIG. 22.

As illustrated in FIG. 22, in the sensor part 50, a pair of thermistor electrodes 133 and 134 are additionally provided so as to contact the thermistor resistor 130 provided on the substrate 110, and a pair of thermistor electrodes 233 and 234 are additionally provided so as to contact the thermistor resistor 230 provided on the substrate 210. The thermistor resistor 130 and the pair of thermistor electrodes 133 and 134 constitute the thermistor Rd4 illustrated in FIG. 21. The thermistor resistor 230 and the pair of thermistor electrodes 233 and 234 constitute the thermistor Rd3 illustrated in FIG. 21.

The distance W4 between the thermistor electrodes 133 and 134 is, for example, 5 to 8 μm and may be selected according to the target resistance value of the thermistor Rd1. The resistance value of the thermistor Rd4 may be substantially the same as that of the thermistor Rd1. The thermistor electrode 133 is connected to a terminal electrode 401 that receives the ground potential GND, and the thermistor electrode 134 is connected to a terminal electrode 402 that constitutes the node N7.

The distance W3 between the thermistor electrodes 233 and 234 is, for example, 5 to 8 μm and may be selected according to the target resistance value of the thermistor Rd3. The resistance value of the thermistor Rd3 may be substantially the same as that of the thermistor Rd2. The thermistor electrode 233 is connected to a terminal electrode 403 that constitutes the node N7. The thermistor electrode 234 is connected to a terminal electrode 404 that receives the power supply potential Vcc.

The thermistors Rd1 and Rd4 are disposed so as not to overlap the heater MH1 on the substrate 110. Thus, the thermistors Rd1 and Rd4 are not heated directly by the heater MH1, but is mainly heated by thermal conduction through the substrate 110. The distance between the heater MH1 and the thermistor Rd1 and the distance between the heater MH1 and the thermistor Rd4 are substantially the same. Thus, when the heater MH1 is heated, the thermistors Rd1 and Rd4 are heated to substantially the same temperature.

The thermistors Rd2 and Rd4 are disposed so as not to overlap the heater MH2 on the substrate 210. Thus, the thermistors Rd2 and Rd3 are not heated directly by the heater MH2, but is mainly heated by thermal conduction through the substrate 210. The distance between the heater MH2 and the thermistor Rd2 and the distance between the heater MH2 and the thermistor Rd3 are substantially the same. Thus, when the heater MH2 is heated, the thermistors Rd2 and Rd3 are heated to substantially the same temperature.

As illustrated in FIG. 21, in the present embodiment, the gas detection signal Vgas1 appearing at the selection node S11 between the thermistor Rd1 and the thermistor Rd2 which are connected in series in this order between the power supply Vcc and the ground GND is supplied to the non-inversion input terminal (+) of the differential amplifier 29, and the gas detection signal Vgas2 appearing at the node N7 between the thermistor Rd4 and the thermistor Rd3 which are connected in series in this order between the power supply Vcc and the ground GND is supplied to the inversion input terminal (-) of the differential amplifier 29. The differential amplifier 29 compares the gas detection signal Vgas1 with the gas detection signal Vgas2 to generate the amplification signal Vamp0 corresponding to the amplified level difference (= Vgas1 – Vgas2) between the gas detection signals Vgas1 and Vgas2.

As described above, in the present embodiment, the thermistors Rd1 to Rd4 are connected in a full bridge, so that the amplification signal Vamp1 exhibits a larger change in accordance with the concentration of CO₂ gas. This makes it possible to further enhance the detection sensitivity for the concentration of CO₂ gas.

### <Fifth Embodiment>

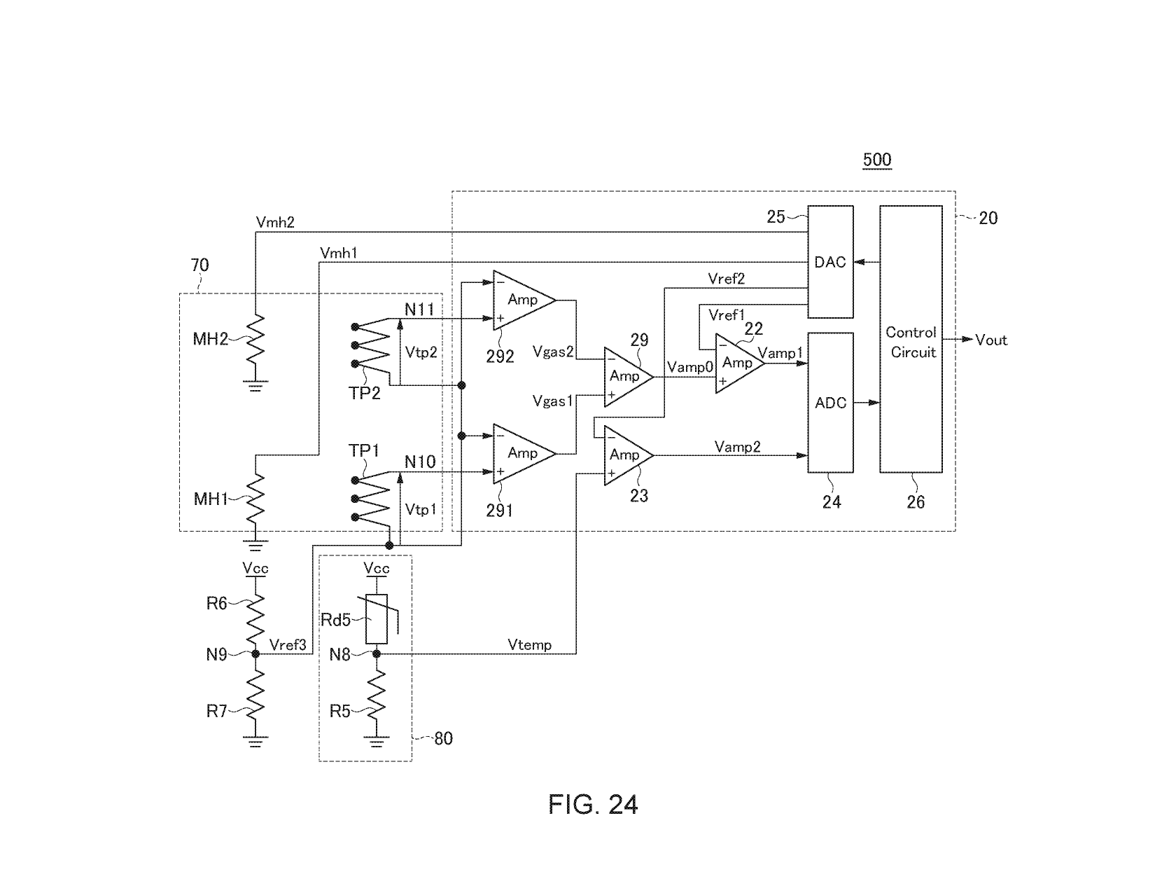

FIG. 24 is a circuit diagram illustrating the configuration of a gas sensor 500 according to a fifth embodiment of the technology described herein.

As illustrated in FIG. 24, the gas sensor 500 according to the fifth embodiment differs from the gas sensor 300 according to the third embodiment in the following points: the sensor part 40 is replaced with a sensor part 70; a temperature sensor 80 is additionally provided; and the multiplexer 28 is removed from the signal processing circuit 20, and instead, differential amplifiers 291 and 292 are provided. Other basic configurations are the same as those of the gas sensor 300 according to the third embodiment, so the same reference numerals are given to the same elements, and overlapping description will be omitted.

The sensor part 70 includes thermopile elements TP1 and TP2 and the heaters MH1 and MH2. The hot junction of the thermopile element TP1 changes its temperature in accordance with variations in the temperature of the heater MH1, and the hot junction of the thermopile element TP2 changes its temperature in accordance with variations in the temperature of the heater MH2. The thermopile elements TP1 and TP2 are each a temperature-sensitive element in which a potential difference appearing between both ends thereof varies depending on temperature. The potential difference appearing between both ends of the thermopile element TP1 is used as an output signal Vtp1, and the potential difference appearing between both ends of the thermopile element TP2 is used as an output signal Vtp2. A reference potential Vref3 is generated by fixed resistors R6 and R7. The fixed resistors R6 and R7 are connected in series between the power supply Vcc and the ground GND, and the reference potential Vref3 appears at a node N9 between the fixed resistors R6 and R7. The reference potential Vref3 is supplied in common to the inversion input terminals (-) of the differential amplifiers 291 and 292 included in the signal processing circuit 20. The output signal Vtp1 is supplied to the non-inversion input terminal (+) of the differential amplifier 291 included in the signal processing circuit 20, and the output signal Vtp2 is supplied to the non-inversion input terminal (+) of the differential amplifier 292 included in the signal processing circuit 20.

The potential supplied to the non-inversion input terminal (+) of the differential amplifier 291 has a level obtained by the output signal Vtp1 corresponding to the temperature-dependent electromotive force of the thermopile element TP1 on the reference potential Vref1. The potential supplied to the non-inversion input terminal (+) of the differential amplifier 292 has a level obtained by the output signal Vtp2 corresponding to the temperature-dependent electromotive force of the thermopile element TP2 on the reference potential Vref3.

The output signal Vtp1 is amplified by the differential amplifier 291 included in the signal processing circuit 20 to generate the gas detection signal Vgas1. The differential amplifier 291 compares the reference potential Vref3 supplied to the inversion input terminal (-) thereof with the level of (Vref3 + Vtp1) supplied to the non-inversion input terminal (+) thereof to generate the gas detection signal Vgas1 corresponding to the amplified level difference (= Vtp1) between the reference potential Vref3 and (reference potential Vref3 + output signal Vtp1).

The output signal Vtp2 is amplified by the differential amplifier 292 included in the signal processing circuit 20 to generate the gas detection signal Vgas2. The differential amplifier 292 compares the reference potential Vref3 supplied to the inversion input terminal (-) thereof with the level of (Vref3 + Vtp2) supplied to the non-inversion input terminal (+) thereof to generate the gas detection signal Vgas2 corresponding to the amplified level difference (= Vtp2) between the reference potential Vref3 and (reference potential Vref3 + output signal Vtp2).

As in the third embodiment, the differential amplifier 29 compares the gas detection signal Vgas1 with the gas detection signal Vgas2 to generate an amplification signal Vamp0 corresponding to the amplified level difference (= Vgas1 – Vgas2) between the gas detection signal Vgas1 and the gas detection signal Vgas2. The amplification signal Vamp0 is supplied to the differential amplifier 22. The differential amplifier 22 compares the amplification signal Vamp0 with the reference potential Vref1 to generate the amplification signal Vamp1 corresponding to the amplified level difference (= Vamp0 – Vref1) between the amplification signal Vamp0 and the reference potential Vref1.

The temperature sensor 80 is a circuit configured to detect the ambient temperature and includes a thermistor Rd5 and a fixed resistor R5 which are connected in series between the power supply Vcc and ground GND. The temperature detection signal Vtemp output from the temperature sensor 80 appears at a node N8 between the thermistor Rd5 and the fixed resistor R5. The temperature sensor 80 may be designed so as not to be affected or so as to be hardly affected by heating by, for example, the heaters MH1 and MH2. The temperature detection signal Vtemp is supplied to the differential amplifier 23 included in the signal processing circuit 20.

Even with such a circuit configuration, when the heater MH1 is heated to the first temperature range, for example, about 170°C during the gas concentration measurement, an increase in the CO₂ gas concentration in the measurement atmosphere causes the temperature of the heater MH1 to rise, whereby the temperature at the hot junction of the thermopile element TP1 also rises in accordance therewith, so that the level of the gas detection signal Vgas1 corresponding to the thermal electromotive force of the thermopile element TP1 increases. On the other hand, when the heater MH2 is heated to the second temperature range, for example, about 340°C during the gas concentration measurement, the temperature of the heater MH2 and the temperature at the hot junction of the thermopile element TP2 hardly rise even if the CO₂ gas concentration in the measurement atmosphere increases, so that the level of the gas detection signal Vgas2 corresponding to the thermal electromotive force of the thermopile element TP2 hardly increases. Such a level difference between the gas detection signals Vgas1 and Vgas2 is amplified by the differential amplifier 29 to generate the amplification signal Vamp0.

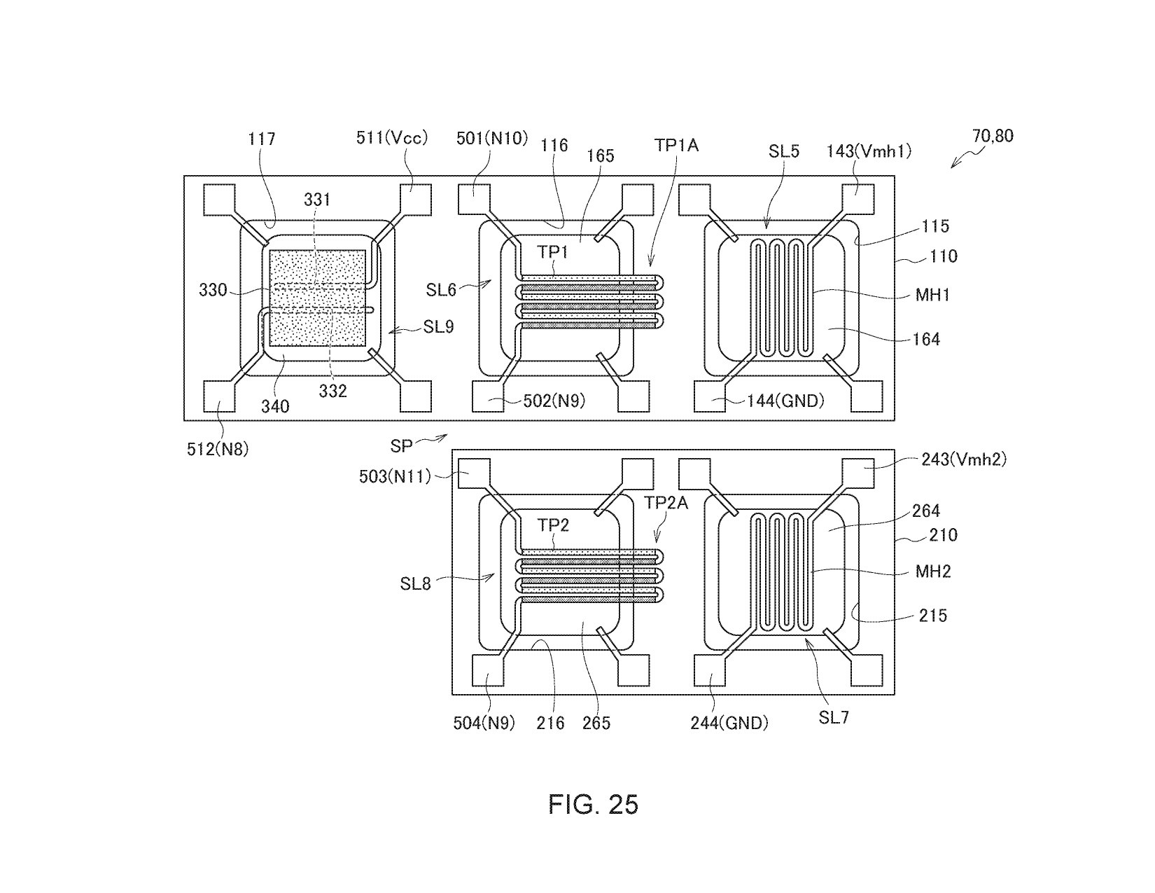

FIG. 25 is a schematic plan view illustrating the configurations of the sensor part 70 and the temperature sensor 80.

In the example illustrated in FIG. 25, the heater MH1, the thermopile element TP1, and the temperature sensor 80 are supported on the substrate 110, and the heater MH2 and the thermopile element TP2 are supported on the substrate 210.

The substrate 110 has three cavities 115 to 117. A slit SL5 is formed in a region overlapping the cavity 115, and the heater MH1 is supported by a membrane 164. A slit SL6 is formed in a region overlapping the cavity 116, and the thermopile element TP1 is partially supported by a membrane 165. The heater MH1 and the thermopile element TP1 are arranged in the X-direction. The heater MH1 side end portion of the thermopile element TP1 constitutes a hot junction TP1A. One end (terminal) of the thermopile element TP1 is connected to a terminal electrode 501 constituting a node N10, and the other end (terminal) thereof is connected to a terminal electrode 502 constituting the node N9.

A slit SL9 is formed in a region overlapping the cavity 117, and the thermistor resistor 330 and a pair of thermistor electrodes 331 and 332 contacting the thermistor resistor 330 are supported by a membrane 340. The thermistor resistor 330 and the pair of thermistor electrodes 331 and 332 constitute the temperature sensor 80 illustrated in FIG. 24. The thermistor electrode 331 is connected to a terminal electrode 511 that receives the power supply potential Vcc, and the thermistor electrode 332 is connected to a terminal electrode 512 that constitutes the node N8.

In the example illustrated in FIG. 25, the thermopile element TP1 is disposed between the heater MH1 and the temperature sensor 80, whereby heat from the heater MH1 is less likely to be conducted to the temperature sensor 80 than to the thermopile element TP1.

The substrate 210 has two cavities 215 and 216. A slit SL7 is formed in a region overlapping the cavity 215, and the heater MH2 is supported by a membrane 264. A slit SL8 is formed in a region overlapping the cavity 216, and the thermopile element TP2 is partially supported by a membrane 265. The heater MH2 and the thermopile element TP2 are arranged in the X-direction. The heater MH2 side end portion of the thermopile element TP2 constitutes a hot junction TP2A. One end (terminal) of the thermopile element TP2 is connected to a terminal electrode 503 constituting a node N11, and the other end (terminal) thereof is connected to a terminal electrode 504 constituting the node N9.

As described above, in the present embodiment, the temperature of the thermopile element TP1 changes due to heat conducted from the heater MH1, mainly through the substrate 110, and in accordance with a change in the temperature of the heater MH1, and the temperature of the thermopile element TP2 changes due to heat conducted from the heater MH2, mainly through the substrate 210, and in accordance with a change in the temperature of the heater MH2.

As exemplified by the gas sensor 500 according to the fifth embodiment, it is not essential to use the thermistor as a temperature-sensitive element; instead, other types of temperature-sensitive elements, such as a thermopile element, may be employed. Further, as exemplified by the gas sensor 500 according to the fifth embodiment, the concentration of a gas to be measured may be calculated based on the difference between the output voltage (gas detection signal Vgas1) derived from the thermopile element TP1 and the output voltage (gas detection signal Vgas2) derived from the thermopile element TP2.

While some embodiments of the technology according to the present disclosure have been described, the technology according to the present disclosure is not limited to the above embodiments, and various modifications may be made within the scope of the present disclosure, and all such modifications are included in the technology according to the present disclosure.

The technology according to the present disclosure includes the following configuration examples, but not limited thereto.

A gas sensor according to an aspect of the present disclosure includes: a substrate having a first cavity; a first heater supported on the substrate so as to overlap the first cavity; a first temperature-sensitive element supported on the substrate so as not to overlap the first heater; and a signal processing circuit configured to heat the first heater during gas concentration measurement, wherein a temperature of the first temperature-sensitive element during the gas concentration measurement changes due to heat conducted from the first heater, mainly through the substrate, and in accordance with a change in a temperature of the first heater. This suppresses the temperature rise of the first temperature-sensitive element during the gas concentration measurement, thereby making it possible to suppress aging of the first temperature-sensitive element.

In the above gas sensor, the substrate may further include a second cavity disposed at a planar position different from a planar position of the first cavity, and the first temperature-sensitive element may be supported on the substrate so as to overlap the second cavity. This enhances the temperature responsiveness of the first temperature-sensitive element.

In the above gas sensor, the first temperature-sensitive element may be supported on the substrate so as to overlap the first cavity. This enhances the temperature responsiveness of the first temperature-sensitive element and enables downsizing of the substrate.

In the above gas sensor, the signal processing circuit may be configured to heat the first heater based on an output value from the first temperature-sensitive element obtained before the first heater is heated. Thus, the first temperature-sensitive element also functions as a temperature sensor, thereby eliminating the need for an additional temperature sensor.

The above gas sensor may further include a second heater and a second temperature-sensitive element, the substrate may further have a second cavity that is disposed at a planar position different from a planar position of the first cavity, the second heater may be supported on the substrate so as to overlap the second cavity, the second temperature-sensitive element may be supported on the substrate so as to overlap neither the first heater nor the second heater, the signal processing circuit may be configured to heat, during the gas concentration measurement, the second heater to a temperature different from the temperature of the first heater, and a temperature of the second temperature-sensitive element during the gas concentration measurement changes due to heat conducted from the second heater, mainly through the substrate, and in accordance with a change in a temperature of the second heater. This makes it possible to selectively detect the concentration of a gas to be measured while reducing the influence of gases other than the gas to be measured and to suppress the temperature rise of the second temperature-sensitive element during the gas concentration measurement. As a result, aging of the second temperature-sensitive element can be suppressed.

In the above gas sensor, the first temperature-sensitive element and the second temperature-sensitive element may be connected in series, and the signal processing circuit may be configured to calculate, during the gas concentration measurement, a concentration of a gas to be measured based on a detection voltage appearing at a node between the first temperature-sensitive element and the second temperature-sensitive element. This allows a half-bridge circuit including the first and second temperature-sensitive elements to obtain a gas detection signal.

In the above gas sensor, the signal processing circuit may be configured to calculate, during the gas concentration measurement, a concentration of a gas to be measured based on a voltage corresponding to a difference between a first output voltage derived from the first temperature-sensitive element and a second output voltage derived from the second temperature-sensitive element. Thus, it is possible to adjust the level of the second output voltage irrespective of the first temperature-sensitive element and to adjust the level of the first output voltage irrespective of the second temperature-sensitive element.

In the above gas sensor, the substrate may include a first substrate having the first cavity and supporting thereon the first heater and the first temperature-sensitive element and a second substrate having the second cavity and supporting thereon the second heater and the second temperature-sensitive element, and the first substrate and the second substrate may be disposed with a space provided therebetween. This makes it possible to prevent thermal interface between the first heater and the second temperature-sensitive element and between the second heater and the first temperature-sensitive element.

In the above gas sensor, the substrate may include a first substrate having the first cavity and supporting thereon the first heater and the first temperature-sensitive element and a second substrate having the second cavity and supporting thereon the second heater and the second temperature-sensitive element, the first substrate may further have a third cavity disposed at a planar position different from the planar position of the first cavity, the second substrate may further have a fourth cavity disposed at a planar position different from the planar position of the second cavity, the first temperature-sensitive element may be supported on the first substrate so as to overlap the third cavity, and the second temperature-sensitive element may be supported on the second substrate so as to overlap the fourth cavity. This enhances the temperature responsiveness of the first and second temperature-sensitive elements.

In the above gas sensor, the first temperature-sensitive element may be supported on the substrate so as to overlap the first cavity, and the second temperature-sensitive element may be supported on the substrate so as to overlap the second cavity. This enhances the temperature responsiveness of the first and second temperature-sensitive elements and enables downsizing of the substrate.

In the above gas sensor, the signal processing circuit may be configured to heat the first heater and the second heater based on at least either an output value, which is derived from the first temperature-sensitive element and obtained before the first heater is heated or an output value, which is derived from the second temperature-sensitive element and obtained before the second heater is heated. Thus, the first and second temperature-sensitive elements also function as temperature sensors, thereby eliminating the need for an additional temperature sensor.

In the above gas sensor, the signal processing circuit may be configured to heat the first heater based on the output value, which is derived from the first temperature-sensitive element and obtained before the first heater is heated and heat the second heater based on the output value, which is derived from the second temperature-sensitive element and obtained before the second heater is heated. This eliminates the need for an additional temperature sensor and enables more accurate measurement of the ambient temperature.

In the above gas sensor, a distance between the second temperature-sensitive element and the second heater may be larger than a distance between the first temperature-sensitive element and the first heater, and the signal processing circuit may be configured to heat, during the gas concentration measurement, the second heater to a temperature higher than the temperature of the first heater. This makes it possible to reduce the difference between the temperature of the first temperature-sensitive element when the first heater is heated and the temperature of the second temperature-sensitive element when the second heater is heated.

In the above gas sensor, each of the first temperature-sensitive element and the second temperature-sensitive element may have a resistor and a pair of electrodes connected to the resistor, and a ratio of a resistance value of the first temperature-sensitive element between the pair of electrodes to a resistance value of the second temperature-sensitive element between the pair of electrodes in a same temperature may be in the range of 0.9 to 1.1. This enables a wide dynamic range.

What is claimed is:

1. A gas sensor comprising:
a substrate having a first cavity;
a first heater supported on the substrate so as to overlap the first cavity;
a first temperature-sensitive element supported on the substrate so as not to overlap the first heater; and
a signal processing circuit configured to heat the first heater during gas concentration measurement,
wherein a temperature of the first temperature-sensitive element during the gas concentration measurement changes due to heat conducted from the first heater, mainly through the substrate, and in accordance with a change in a temperature of the first heater.

2. The gas sensor as claimed in claim 1,
wherein the substrate further includes a second cavity disposed at a planar position different from a planar position of the first cavity, and
wherein the first temperature-sensitive element is supported on the substrate so as to overlap the second cavity.

3. The gas sensor as claimed in claim 1, wherein the first temperature-sensitive element is supported on the substrate so as to overlap the first cavity.

4. The gas sensor as claimed in claim 1, wherein the signal processing circuit is configured to heat the first heater based on an output value from the first temperature-sensitive element obtained before the first heater is heated.

5. The gas sensor as claimed in claim 1, further comprising:
a second heater; and
a second temperature-sensitive element,
wherein the substrate further has a second cavity that is disposed at a planar position different from a planar position of the first cavity,
wherein the second heater is supported on the substrate so as to overlap the second cavity,
wherein the second temperature-sensitive element is supported on the substrate so as to overlap neither the first heater nor the second heater,
wherein the signal processing circuit is configured to heat, during the gas concentration measurement, the second heater to a temperature different from the temperature of the first heater, and
wherein a temperature of the second temperature-sensitive element during the gas concentration measurement changes due to heat conducted from the second heater, mainly through the substrate, and in accordance with a change in a temperature of the second heater.

6. The gas sensor as claimed in claim 5,
wherein the first temperature-sensitive element and the second temperature-sensitive element are connected in series, and
wherein the signal processing circuit is configured to calculate, during the gas concentration measurement, a concentration of a gas to be measured based on a detection voltage appearing at a node between the first temperature-sensitive element and the second temperature-sensitive element.

7. The gas sensor as claimed in claim 5, wherein the signal processing circuit is configured to calculate, during the gas concentration measurement, a concentration of a gas to be measured based on a voltage corresponding to a difference between a first output voltage derived from the first temperature-sensitive element and a second output voltage derived from the second temperature-sensitive element.

8. The gas sensor as claimed in claim 5,
wherein the substrate includes a first substrate having the first cavity and supporting thereon the first heater and the first temperature-sensitive element and a second substrate having the second cavity and supporting thereon the second heater and the second temperature-sensitive element, and
wherein the first substrate and the second substrate are disposed with a space provided therebetween.

9. The gas sensor as claimed in claim 5,
wherein the substrate includes a first substrate having the first cavity and supporting thereon the first heater and the first temperature-sensitive element and a second substrate having the second cavity and supporting thereon the second heater and the second temperature-sensitive element,
wherein the first substrate further has a third cavity disposed at a planar position different from the planar position of the first cavity,
wherein the second substrate further has a fourth cavity disposed at a planar position different from the planar position of the second cavity,
wherein the first temperature-sensitive element is supported on the first substrate so as to overlap the third cavity, and
wherein the second temperature-sensitive element is supported on the second substrate so as to overlap the fourth cavity.

10. The gas sensor as claimed in claim 5,
wherein the first temperature-sensitive element is supported on the substrate so as to overlap the first cavity, and
wherein the second temperature-sensitive element is supported on the substrate so as to overlap the second cavity.

11. The gas sensor as claimed in claim 5, wherein the signal processing circuit is configured to heat the first heater and the second heater based on at least either an output value, which is derived from the first temperature-sensitive element and obtained before the first heater is heated or an output value, which is derived from the second temperature-sensitive element and obtained before the second heater is heated.

12. The gas sensor as claimed in claim 11, wherein the signal processing circuit is configured to heat the first heater based on the output value, which is derived from the first temperature-sensitive element and obtained before the first heater is heated and heat the second heater based on the output value, which is derived from the second temperature-sensitive element and obtained before the second heater is heated.

13. The gas sensor as claimed in claim 5,
wherein a distance between the second temperature-sensitive element and the second heater is larger than a distance between the first temperature-sensitive element and the first heater, and
wherein the signal processing circuit is configured to heat, during the gas concentration measurement, the second heater to a temperature higher than the temperature of the first heater.

14. The gas sensor as claimed in claim 5,
wherein each of the first temperature-sensitive element and the second temperature-sensitive element has a resistor and a pair of electrodes connected to the resistor, and
wherein a ratio of a resistance value of the first temperature-sensitive element between the pair of electrodes to a resistance value of the second temperature-sensitive element between the pair of electrodes in a same temperature is in a range of 0.9 to 1.1.
